SEQUENCE LISTING (12 sequences)

SEQ ID NO: 1 - protein, 11 aa, Artificial Sequence
  Synthetic peptide
RMSEIKRLLSE

SEQ ID NO: 2 - protein, 11 aa, Artificial Sequence
  Synthetic peptide
RLRTLRRLLQE

SEQ ID NO: 3 - protein, 13 aa, Artificial Sequence
  Synthetic peptide
RLEPNDIVYLIRS

SEQ ID NO: 4 - protein, 12 aa, Artificial Sequence
  Synthetic peptide
RASDKQTLLPNQ

SEQ ID NO: 5 - protein, 12 aa, Artificial Sequence
  Synthetic peptide
RASDKQTLLPNQ

SEQ ID NO: 6 - protein, 11 aa, Artificial Sequence
  Synthetic peptide
IERLSEMSLRK

SEQ ID NO: 7 - protein, 6 aa, Artificial Sequence
  Synthetic peptide
  MISC_FEATURE (1)..(1): X is D, E, S, or T
  MISC_FEATURE (2)..(2): X is any amino acid
  MISC_FEATURE (3)..(3): X is any amino acid
  MISC_FEATURE (4)..(4): X is any amino acid
  MISC_FEATURE (6)..(6): X is L or I
XXXXLX

SEQ ID NO: 8 - protein, 7 aa, Artificial Sequence
  Synthetic peptide
  MISC_FEATURE (1)..(1): X is S or T
  MISC_FEATURE (2)..(2): X is D, E, S, or T
  MISC_FEATURE (3)..(3): X is any amino acid
  MISC_FEATURE (4)..(4): X is any amino acid
  MISC_FEATURE (5)..(5): X is any amino acid
  MISC_FEATURE (7)..(7): X is L or I
XXXXXLX

SEQ ID NO: 9 - protein, 6 aa, Artificial Sequence
  Synthetic peptide
EIKRLL

SEQ ID NO: 10 - protein, 6 aa, Artificial Sequence
  Synthetic peptide
TLRRLL

SEQ ID NO: 11 - protein, 6 aa, Artificial Sequence
  Synthetic peptide
DIVYLI

SEQ ID NO: 12 - protein, 6 aa, Artificial Sequence
  Synthetic peptide
DKQTLL

(19) United States
(12) Patent Application Publication
(10) Pub. No.: US 2024/0294571 A1
(43) Pub. Date: Sep. 5, 2024

(54) ANALGESIC AND ANESTHETIC PEPTIDES AND OTHER AGENTS

(71) Applicant: The Research Foundation for The State University of New York, Amherst, NY (US)

(72) Inventor: Arindam BHATTACHARJEE, Buffalo, NY (US)

(21) Appl. No.: 17/768,142

(22) PCT Filed: Oct. 12, 2020
(86) PCT No.: PCT/US2020/055289

§ 371 Date: Apr. 11, 2022

Related U.S. Application Data
(60) Provisional application No. 62/913,512, filed on Oct. 10, 2019.

(51) Int. Cl. C07K 7/06; A61K 45/06; A61P 23/00; A61P 29/00; C07K 7/08

(52) U.S. Cl. CPC C07K 7/06; A61K 45/06; A61P 23/00; A61P 29/00; C07K 7/08

(57) ABSTRACT

Disclosed are agents and methods of using the agents to treat or prevent pain and/or induce anesthesia. The agents may be peptides, siRNAs, and/or shRNAs that target adaptin protein 2-clathrin mediated endocytosis (AP2-CME). Peptides of the present disclosure may contain the following sequence X¹X²X³X⁴LX⁵ (SEQ ID NO:7), where X¹ is D, E, S, or T, where the D, E, S, and/or the T is optionally phosphorylated, X², X³, and X³ are independently chosen from any amino acid.

### CROSS REFERENCE TO RELATED APPLICATIONS

This application is the National Stage Entry of PCT/US2020/055289, filed on Oct. 12, 2020, which claims priority to U.S. Provisional Application No. 62/913,512, filed on Oct. 10, 2019, the disclosure of which is incorporated by reference herein.

### STATEMENT REGARDING FEDERALLY SPONSORED RESEARCH

This invention was made with government support under grant no. NS108087 awarded by the National Institutes of Health. The government has certain rights in the invention.

### SEQUENCE LISTING

The instant application contains a Sequence Listing which has been submitted electronically in ASCII format and is hereby incorporated by reference in its entirety. Said ASCII copy, created on Oct. 12, 2020, is named “011520_01537_SEQ_ID_ST25.txt”, and is 3,373 bytes in size.

### BACKGROUND OF THE DISCLOSURE

The physiology of inflammatory pain involves the integration of primary afferent neurons, the central nervous system, and the immune system. Peripheral sensitization of dorsal root ganglion (DRG) nociceptors initiates inflammatory pain and is driven by inflammatory mediators released from immune cells and damaged tissue. Recently, calcitonin gene-related peptide (CGRP) containing nociceptors were identified as principal coordinators of thermal and mechanical sensitivity in various pain models. Therefore, it is reasonable to consider CGRP⁺ nociceptors as potential analgesic targets.

There is an unmet need for efficacious analgesics with lesser adverse effects. Opioid drugs, the most widely prescribed class of medications in the United States, are commonly used for pain treatment. In addition to their high potential for addiction, there are concerns that opioids can lead to hypotension, sleep apnea, reduced hormone production and, in the elderly, increased falls and hip fractures. Opioids also cause respiratory depression, and there is now an ever-increasing concern over the intersection of the opioid epidemic with the Covid-19 pandemic. Other treatment options for inflammatory pain include non-steroidal anti-inflammatory drugs and corticosteroids, but they have been increasingly contraindicated for extended use due to detrimental side effects. Nociceptive ion channel inhibitors seemed to be attractive molecules for analgesia, however, they have demonstrated limited clinical efficacy and are not currently used as a treatment option. After screening more than three-thousand transgenic mouse knockout lines, the endocytosis associated-adaptin protein kinase 1 (AAK1), was considered as a putative target for pain treatment and small molecules were developed to inhibit this enzyme. Targeting AAK1 systemically, however, might be problematic due to its ubiquitous expression and further development of AAK1 inhibitors for pain relief has yet to be pursued. Nonetheless, this study did mark the first pre-clinical attempt to provide analgesia by pharmacologically inhibiting endocytosis.

The primary endocytic machinery in neurons utilizes the multimeric adaptor protein complex 2 (AP2), which has differential expression of its α-subunit isoforms: the α1 isoform localizes to synaptic compartments, whereas the α2 isoform exhibits robust extra-synaptic expression. AP2 clathrin-mediated endocytosis (AP2-CME) was shown to underlie DRG neuronal sensitization through internalization of sodium-activated potassium channels (K_{Na}) in vitro and that the AP2α2 subunit becomes associated with these channels after protein kinase A (PKA) stimulation.

### SUMMARY OF THE DISCLOSURE

The present disclosure provides agents and methods of using these agents to treat or prevent pain and/or induce anesthesia. The agents are peptides, siRNAs, and/or shRNAs targeting adaptin protein 2-clathrin mediated endocytosis (AP2-CME). In one aspect, use of these agents will diminish or eliminate the need for narcotics (e.g., opioids) to combat pain.

### BRIEF DESCRIPTION OF THE FIGURES

For a fuller understanding of the nature and objects of the disclosure, reference should be made to the following detailed description taken in conjunction with the accompanying figures.

FIG. 1 depicts the genetic knockdown of AP2A2 subunit attenuating acute inflammatory pain-like behaviors in mice. Panel A presents summarized pain-like behaviors from C57BL/6 mice following injection with 5% formalin. Phase 1 was 0-10 minutes post injection and phase 2 was 11-60 minutes post-injection (scrambled shRNA group n=6; AP2A2 group n=6). The displayed data is presented as cumulative means±s.e.m. The significance, * (p<0.05), ** (p<0.01), was determined using a 2-way ANOVA with a Bonferroni correction. Panel B provides representative images depicting pain-like behaviors in C57BL/6 mice at 2 minutes on the left side, at 20 minutes in the middle and 60 minutes on the right side, post-formalin injection. The arrow, shown in the bottom right of Panel B, highlights the use of inflamed paw in the mouse with depleted AP2A2. Illustrated at the top of Panel C are representative western blots showing the extent of AP2A2 knockdown. The representative western blots are paired contralateral and ipsilateral samples taken from the same animal. Depicted at the bottom of Panel C is the densitometry analysis of western blots (n=3). The data is presented as mean±s.e.m. The significance, * (p<0.05), was determined using a 2-way ANOVA with a Bonferroni correction.

FIG. 2 illustrates the knockdown of AP2A2 impacts on the initiation and maintenance of thermal sensitivity in chronic inflammatory pain models. The top portion of Panel A is a timeline highlighting time points for chronic inflammatory pain in a pre-emptive knockdown model. The bottom portion of Panel A shows the summarized data from the Hargreaves assay. Contralateral and ipsilateral paw withdrawal latencies for scrambled (n=11) and AP2A2 (n=12) shRNA groups are shown. The displayed data is represented as the mean±s.e.m. The significance, * (p<0.05), ** (p<0.01), was determined using the 2-way ANOVA with a Bonferroni correction. The top portion of Panel B is a timeline noting time points for chronic in an inflammatory pain post facto knockdown model. The bottom portion of Panel B shows the summarized data from the Hargreaves assay. Contralateral and ipsilateral paw withdrawal latencies for scrambled (n=8) and AP2α2 (n=8) shRNA groups are shown. The data is represented as the mean±s.e.m. The significance, * (p<0.05), ** (p<0.01), was determined using the 2-way ANOVA with a Bonferroni correction.

FIG. 3 shows interplantar injection of the AP2 inhibitor peptide attenuating acute and chronic inflammatory pain behaviors in mice. Panel A provides the summarized pain-like behaviors from C57BL/6 mice following injection with 5% formalin. Shown are phase 1, which is 0-10 minutes post-injection, and phase 2, which is 11-60 minutes post-injection (scrambled peptide group n=6; AP2 inhibitor peptide group n=6). The displayed data is presented as cumulative means±s.e.m. The significance, * (p<0.05), was determined using 2-way ANOVA with a Bonferroni correction. Panel B shows the summarized data from the Hargreaves assay. Contralateral and ipsilateral paw withdrawal latencies for scrambled and AP2 inhibitor peptide groups are shown. The data is represented as mean±s.e.m. The significance, * (p<0.05), ** (p<0.01), *** (0.005), was determined using the 2-way ANOVA. Panel C depicts the summarized data from the Von Frey assay. The data is presented as mean force required to illicit the paw withdrawal response±s.e.m. The significance, * (p<0.05), was determined using multiple t-tests.

FIG. 4 illustrates a proposed mechanism for peptide inhibition of the AP2 Complex. It is proposed that a lipidated peptide enters the cell by a flip-flop mechanism. Once inside the cell, the peptide remains tethered to the inner leaflet of the plasma membrane. Then, the peptide (dileucine-based) interacts with the AP2 complex. The interaction prevents the AP2 complex from binding its substrates and inhibiting endocytosis.

FIG. 5 depicts peptide analogs that partially attenuate acute inflammatory pain behaviors in mice. The summarized pain-like behaviors from C57BL/6 mice following injection with 5% formalin are shown. Two phases are depicted—phase 1 represents 0-10 minutes post-injection and phase 2 represents 11-60 minutes post-injection (scrambled peptide group n=6; AP2 inhibitor peptide group n=6; P1 group n=3; P2 group n=3; P3 group n=3, P4 group n=3). The P4 peptide, which is a phosphorylated variant of the P3 peptide, was the only peptide that showed significant decreases in both licking and lifting behaviors. The data is presented as cumulative means±s.e.m. The significance, * (p<0.05), was determined using multiple t-tests.

FIG. 6 demonstrates that ablation of the AP2-mediated endocytosis did not impact edema in the ipsilateral hind paw. Shown in Panel A is a summarized cross-sectional area from the ipsilateral paw of C57BL/6 mice 24-hours before and after injection of CFA; 7 days post-shRNA nerve injection. The measurements were taken with a caliper from the thickest part of the paw. The data is presented as mean cross-sectional area±s.e.m (n=3). Panel B presents a summarized cross-sectional area from the ipsilateral paw of C57BL/6 mice 24-hours before and after injection of CFA. Both groups were pre-injected with peptide 24 hours before ‘Pre-CFA’ measurement. The measurements were taken with a caliper from the thickest part of the paw. The data presented as mean cross-sectional area±s.e.m (n=3).

FIG. 7 shows AP2α2 is expressed in IB4⁻, CGRP⁺ nociceptors and in vivo AP2α2 knockdown attenuates nocifensive behaviors. (A) Representative immunofluorescent image showing expression patterns of AP2α2 and CGRP. IB4 reactivity was used to delineate between peptidergic and non-peptidergic DRG neurons. AP2α2 is preferentially expressed in small- and medium-sized CGRP⁺ DRG neurons but not in IB4⁺ neurons. Arrows highlight strong co-localization of CGRP and AP2α2 (B) AP2α2 immunoreactivity in the ipsilateral DRG after in vivo AP2α2 knockdown compared to contralateral DRG taken from the same animal, seven days after knockdown. (C) [Left] Representative Western blot showing extent of AP2α2 knockdown. Paired contralateral and ipsilateral samples are taken from the same animal. [Right] Densitometry analysis of Western blots (n=3). Animals were sacrificed seven days after knockdown. Data is presented as mean±s.e.m. Significance determined by one-way ANOVA with Holms-Sidak correction * p<0.05; (D) Representative traces from dissociated adult DRG neurons recorded under varying conditions: [Top] Control conditions, [Middle] DRG neurons transfected with Scrambled shRNAs during PKA stimulatory conditions, [Bottom] DRG neurons transfected with AP2α2 shRNAs during PKA stimulatory conditions. IB4− were selectively recorded as determined by absence of fluorescent after incubation with an IB4-alexa fluor 488 conjugate. (E) Summarized nocifensive behaviors from C57BL/6 mice following injection with 5% formalin. Phase 1; 0-10 minutes, phase 2; 11-60 minutes post-injection (scrambled shRNA group n=6; AP2α2 group n=6). (F) Representative images depicting pain-like behaviors in C57BL/6 mice 2 minutes [left], 20 minutes [middle] and 60 minutes [right] post-formalin injection. Arrow highlights use of inflamed ipsilateral paw. Data is presented as cumulative means±s.e.m. Significance determined using repeated measures 2-way ANOVA with Bonferroni correction p<0.05; *, p<0.01; **

FIG. 8 shows AP2α2 knockdown disrupts the development and maintenance of thermal sensitivity in chronic inflammatory pain. (A) Thermal sensitivity of animals in the CFA pain model with a pre-inflammatory knockdown paradigm for scrambled (n=11) and AP2α2 (n=12) shRNA groups. Data is represented as mean paw withdrawal latency (PWL)±s.e.m. Significance determined using repeated measures 2-way ANOVA with Bonferroni correction p<0.05; *, p<0.01; ** (B) Mechanical sensitivity of ipsilateral hind paw in a chronic inflammatory pain model where knockdown occurred before inflammation was initiated. The data for the scrambled (n=8) and AP2α2 (n=8) shRNA groups is represented as mean percentage of baseline±s.e.m. Significance determined using repeated measures 2-way ANOVA with Bonferroni correction p<0.05; *. Contralateral PWT can be found in FIG. 13. (C) Thermal sensitivity of animals injected with scrambled (n=8) and AP2α2 (n=8) shRNAs following establishment of inflammation. Data is represented as mean PWL s.e.m. Significance determined using repeated measures 2-way ANOVA with Bonferroni correction p<0.05; *, p<0.01; **

FIG. 9 shows lipidated peptides infiltrate peripheral neuronal afferents. (A) Injection of the antigenic lipidated-HA peptide into the hind paw of a naïve C57BL/6 mouse under control conditions. The HA peptide allowed for immunofluorescent visualization of lipidated peptide distribution following injection. [A′]. Heatmap analysis of immunoreactivity [A″ ] The lipidated-HA peptide preferentially partitioned to the dermis, within lipid dense areas. [A1] Insert depicting HA immunoreactivity in peripheral afferents within the dermis. [A2] Insert depicting immunoreactivity in peripheral afferents in muscle tissue. While afferents exhibited immunolabeling, muscle cells did not. (B) Injection of an antigenic lipidated peptidomimetic into the hind paw of a naïve C57BL/6 mouse under CFA-induced inflammation. [B′]. Heatmap analysis depicted greater retention of peptide within inflamed tissues [B″ ] Again, lipidated-HA peptide preferentially partitioned to the dermis, specifically, lipid dense areas. [B1] Insert depicting immunoreactivity in peripheral afferents in the dermis. [B2] Insert depicting immunoreactivity in peripheral afferents and in muscle tissue.

FIG. 10 shows pharmacological inhibition of peripheral endocytosis by a lipidated AP2 inhibitory peptide attenuated pain behaviors during acute and chronic inflammation. (A) Summarized nocifensive behaviors from C57BL/6 mice following injection with 5% formalin. The AP2 inhibitory peptide inhibitor was locally injected into the hind paw 24 hours before formalin injection. Phase 1; 0-10 minutes, phase 2; 15-60 minutes post-injection (scrambled shRNA group n=6; AP2α2 group n=6). Data is presented as mean±s.e.m. Significance determined using repeated measures 2-way ANOVA with Bonferroni correction p<0.05; * (B-D) Thermal sensitivity in animals during established CFA-induced inflammatory pain. Data is represented as mean PWL±s.e.m. Significance determined using repeated measures 2-way ANOVA with Bonferroni correction p<0.05; *, p<0.01; **, p<0.005; *** (B) Composite graph of all animals that received either the scrambled peptide (n=8) or the AP2 inhibitor peptide (n=8). Each group was injected with the respective peptide 24 hours after CFA. (C) Thermal sensitivity of male animals injected with scrambled (n=4) and AP2 inhibitor (n=4) peptide after CFA. Males exhibited immediate return to baseline after AP2 inhibitor peptide injection. (D) Thermal sensitivity of female animals injected with scrambled (n=4) and AP2 inhibitor (n=4) peptide groups. Females injected with AP2 inhibitor peptide showed accelerated recovery compared to scrambled peptide but exhibited a delay compared to male mice (E-G) Total area under the curve (A.U.C.) quantification for each experimental condition. Data is represented are the mean A.U.C. s.e.m. Statistical significance was determined using one-way ANOVA with Holms-Sidak correction p<0.05; *, p<0.01; **, p<0.005; ***, p<0.001; **** (E) Total A.U.C. for all animals under experimental conditions displayed in (B); scrambled peptide (n=8) and AP2 inhibitor peptide (n=8). Pharmacological inhibition of endocytosis generated a significant increase in A.U.C. for the ipsilateral paw. (F) Total A.U.C. for male animals under experimental conditions; scrambled peptide (n=4) and AP2 inhibitor peptide (n=4). Isolating the male subjects from the total data set reveals a retention of the analgesic-like effect observed in (E). (G) Total A.U.C. for female animals under experimental conditions; scrambled peptide (n=4) and AP2 inhibitor peptide (n=4). Interestingly, isolation of the female subjects from the total data set reveals a muted analgesic-like. (H-J) Recovery curves fit to an exponential decay equation. (H) Fitting the recovery curves from (B) reveals that inhibition of endocytosis accelerated the rate of recovery as indicated by a decrease in tau. (I) Recovery curves for males taken from (C) and fit to an exponential decay equation. Males animals responded well to inhibition of endocytosis as indicated by a robust decrease in tau. (J) Recovery curves for females taken from (D) and fit to an exponential decay equation. Female animals did not experience a change in the rate of their recovery following inhibition of endocytosis. (K) Mechanical ipsilateral PWT of animals in either the scrambled peptide (n=11) or the AP2 inhibitor peptide (n=11) groups following establishment of CFA-induced inflammatory pain. Data is represented as mean PWL (as a percentage of baseline PWT)±s.e.m. Significance determined using repeated measures 2-way ANOVA with Bonferroni correction p<0.05; * Contralateral PWT can be found in FIG. 14.

FIG. 11 shows pharmacological inhibition of peripheral endocytosis by a lipidated AP2 inhibitory peptide attenuated thermal sensitivity in a post-operative pain model. (A) Schematic depicting injection protocol for lipidated AP2 inhibitory peptide. (B-D) Thermal sensitivity in animals in a model of post-incisional pain. Data is represented as mean PWL±s.e.m. Significance determined using repeated measures 2-way ANOVA with Bonferroni correction p<0.05; *, p<0.01; **, p<0.005; ***, p<0.001; **** (B) Composite graph depicting thermal sensitivity in animals following plantar muscle incision and injection with scrambled peptide (n=12) or AP2 inhibitory peptide (n=12). (C) Thermal sensitivity of male animals injected with scrambled (n=6) and AP2 inhibitor peptide (n=6) following plantar incision. Males exhibited an early-phase response to local inhibition of endocytosis that was characterized by statistically significant increases in PWT day 1-day 4. (D) Thermal sensitivity of female animals injected with scrambled (n=6) and AP2 inhibitor peptide (n=6) following plantar incision. Females exhibited a late-phase response to local inhibition of endocytosis that was characterized by statistically significant increases in PWT day 3-day 6. (E-G) Total area under the curve (A.U.C.) quantification for each experimental condition. Data is represented are the mean A.U.C. s.e.m. Statistical significance was determined using one-way ANOVA with Holms-Sidak correction p<0.05; *, p<0.01; **, p<0.005; ***, p<0.001; **** (E) Total A.U.C. for all animals under experimental conditions displayed in (B); scrambled peptide (n=12) and AP2 inhibitor peptide (n=12). Pharmacological inhibition of endocytosis generated a significant increase in A.U.C. for the ipsilateral paw. (F) Total A.U.C. for male animals under experimental conditions; scrambled peptide (n=6) and AP2 inhibitor peptide (n=6). Isolating the male subjects from the total data set revealed a trend towards an analgesic-like effect. (G) Total A.U.C. for female animals under experimental conditions; scrambled peptide (n=6) and AP2 inhibitor peptide (n=6). Interestingly, isolation of the female subjects from the total data set reveals a trend towards an analgesic-like effect as well. (H-J) Recovery curves fit to an exponential decay equation. (H) Fitting the recovery curves from (B) reveals that inhibition of endocytosis accelerated the rate of recovery as indicated by a decrease in tau. (I) Recovery curves for males taken from (C) and fit to an exponential decay equation. Males animals responded well to inhibition of endocytosis as indicated by a robust decrease in tau. (J) Recovery curves for females taken from (D) and fit to an exponential decay equation. (K) Mechanical ipsilateral PWT of animals in either the scrambled peptide (n=11) or the AP2 inhibitor peptide (n=11) post-incision. Data is represented as mean PWL (as a percentage of baseline PWT)±s.e.m. Significance determined using repeated measures 2-way ANOVA with Bonferroni correction p<0.05; * Contralateral PWT can be found in FIG. 16.

FIG. 12 shows local inhibition of endocytosis in peripheral nociceptors potentiated CGRP immunoreactivity in the superficial layers of the dermis and Ap2α2 is differentially distributed in CGRP⁺ human DRG neurons. (A) Representative image showing CGRP immunoreactivity in an uninflamed hind paw injected with the scrambled peptide. Typically, CGRP immunoreactivity terminates in the proximal stratum granulosum (SG) (B) Representative image showing CGRP immunoreactivity in an uninflamed hind paw injected with the AP2 inhibitor peptide. White arrows; peripheral nerve fibers exhibited robust CGRP immunoreactivity in the very distal layers of the SG and some CGRP immunoreactive fibers could be seen in very superficial stratum corneum (SC) layer. Yellow arrows; peripheral nerve fibers displaying CGRP immunoreactivity in superficial layers of the SC. (A′ and B′) Magnified sections illustrating SG quadrants. (C) Quantification of CGRP⁺ afferent termination in each SG quadrant (n=3). Significance determined using multiple t-test with p<0.05; * Data is presented as mean s.e.m. (D) Representative immunofluorescent image showing expression patterns of AP2α2 (left) and CGRP (middle) in hDRGs. AP2α2 is differential expressed in CGRP⁺ DRG neurons. Arrows highlight strong co-localization of CGRP and AP2α2.

FIG. 13 shows shRNA mediated knockdown of AP2α2 does not significantly impair CFA-induced ipsilateral swelling or contralateral mechanical behavior. (A) Cross-sectional area of ipsilateral hind paws 24 hours before and following CFA injection. Measurements were taken from awake C57BL/6 mice with calipers. Width and height were taken from the widest part of the hind paw. Each group (scrambled shRNA and AP2α2 shRNA) has n=3. The same animal was measured before and following CFA injection. Data is represented as cross sectional area mean±s.e.m. and analyzed using 2-way ANOVA statistical test with Bonferroni correction p<0.05. (B-D) Von frey filament testing of animals in a CFA-induced model of chronic inflammatory pain. Data is represented as mean PWT (as a percent of baseline PWT)±s.e.m. Statistical significance was determined using repeated measures 2-way ANOVA statistical test with Bonferroni correction p<0.05; * (B) Contralateral PWT. Data is complimentary to data presented in FIG. 2B. (C) [Left] Contralateral PWT for male animals injected with either scrambled shRNA (n=4) or AP2α2 shRNA (n=4). [Right] Ipsilateral paw. (D) [Left] Contralateral PWT for female animals injected with either scrambled shRNA (n=4) or AP2α2 shRNA (n=4). [Right] Ipsilateral paw.

FIG. 14 shows lipidated HA-peptide exhibits robust stability in mitotic cells and post-mitotic neurons. CHO cells were cultured in appropriate conditions for at least 2 days following seeding. On the day of experimentation, the media was changed, and replaced with growth media supplemented with the HA-peptide (10 μM). The cells were incubated in the HA-peptide supplemented media for 3 hours, at which point the media was removed, the cells were washed with PBS and allowed to grow until collection. Cells were fixed and stained with a HA-specific antibody (A) Representative images from cultured CHO cells exposed to the HA-peptide under varying conditions of permeabilization and time points. ‘−Triton x-100’ is indicative of extracellular-only HA-peptide, whereas ‘+Triton x-100’ is indicative of total HA-peptide immunoreactivity. Using permeabilizing and non-permeabilizing conditions demonstrated that the HA-peptide can flip flop continuously from either side of the cell membrane and is therefore primarily membrane-delimited. (B) Representative image of HA immunoreactivity in cultured embryonic DRG neurons 3 days following initial exposure to the HA-peptide. (Bottom) A look-up-table transformation of the top image depicting intensity of staining.

FIG. 15 shows pharmacological inhibition of endocytosis did not alter development of CFA-induced inflammation and mechanical sensitivity. (A) Cross-sectional area of ipsilateral hind paws 24 hours before and 24 hours following CFA injection. Measurements were taken from awake C57BL/6 mice with calipers. Width and height were taken from the widest part of the hind paw. Each group (scrambled and AP2α2 peptidomimetics) has n=3. The same animal was measured before and following CFA injection. Data is represented as cross sectional area mean±s.e.m. and analyzed using 2-way ANOVA statistical test with Bonferroni correction p<0.05. (B-D) Von frey filament testing of animals in a CFA-induced model of chronic inflammatory pain. Data is represented as mean PWT (as a percentage of baseline PWT)±s.e.m. Statistical significance was determined using repeated measures 2-way ANOVA statistical test with Bonferroni correction p<0.05. (B) Contralateral PWT. Data is complimentary to data presented in FIG. 4K. (C) [Left] Contralateral PWT for male animals injected with either scrambled peptide (n=7) or AP2α2 inhibitor peptide (n=7). [Right] Ipsilateral paw. (D) [Left] Contralateral PWT for female animals injected with either scrambled shRNA (n=4) or AP2α2 shRNA (n=4). [Right] Ipsilateral paw.

FIG. 16 shows pharmacological inhibition of endocytosis did not alter contralateral mechanical sensitivity in a model of post-incisional pain. Dynamic Von frey filament testing of animals in a post-incisional model of chronic inflammatory pain. Data is represented as mean PWT (as a percentage of baseline PWT)±s.e.m. Statistical significance was determined using repeated measures 2-way ANOVA statistical test with Bonferroni correction p<0.05. Data is complimentary to data presented in FIG. 11K.

FIG. 17 shows magnitude of early recovery from post-incisional model of pain in scrambled peptide injected animals displays slight sex-dependent trend. Control animals from post-incisional model of pain displayed slight sex differences during early-stage recovery as measured by paw withdrawal latency from a thermal stimulus. Female control animals (n=6) trended towards having a higher paw withdrawal latency 24 hours following incision, while displaying relatively flat rate of recovery. Whereas male control animals (n=6) displayed a lower paw withdrawal latency 24 hours following recovery, that was followed by a linear rate of recovery that matched the female animals at later time points.

FIG. 18 shows efficacy of analgesia is dependent upon peptide sequence. (A) Summarized pain-like behaviors from C57BL/6 mice following injection with 5% formalin. Phase 1; 0-10 minutes, phase 2; 11-60 minutes post-injection (scrambled peptide group n=6; AP2 inhibitory peptide group n=6; P1 group n=3; P2 group n=3; P3 group n=3; P4 group n=3) Peptide sequences found in Table 1. Data is presented as cumulative means±s.e.m. Significance determined using 2-way ANOVA with Bonferroni correction p<0.05; * (B) Summarized thermal sensitivity of rats injected with different Nav1.8 targeted peptidomimetics denoted: Ch1001 (n=10) and Ch1002 (n=10) (from Pryce et al., ref 17). Light gray line denotes AP2 peptidomimetic mean as a reference. All data is represented as mean±s.e.m. and analyzed with 2-way ANOVA with Bonferroni correction, p<0.05; +. ‘+’ Denotes statistical significance between Ch1002 and scrambled groups.

FIG. 19 shows local inhibition of endocytosis did not alter immune cell recruitment but did produce granuloma-like artifacts in an incisional pain model. (A) Animals were treated as previously stated in the Methods section. However, no behavior was collected, instead, animals were sacrificed via transcardial perfusion, and tissue was collected for staining. (Top) Representative images depicting hematoxylin & eosin staining of rat hind paws in scrambled (n=2) and AP2 inhibitory peptide groups (n=2). Localized immune cells can be observed in the dermis in each condition. (Bottom) 24 hours following incision, there is a rapid increase in the number of immune cells. (Bottom right) Inhibition of endocytosis resulted in dense clustering of immune cells. Injection of the peptide did not potentiate any observable changes in gait. (B) Representative images of incision site in animals that received either the scrambled peptide (top) or AP2 inhibitory peptide (bottom). Black arrows highlight significant granuloma-like structures.

FIG. 20 shows inhibition of endocytosis did not significantly change dermal CGRP immunoreactivity in inflamed paws. (A) Representative image showing CGRP immunoreactivity in a 24 hour CFA-induced inflamed hind paw injected with the scrambled peptidomimetic. (B) Representative image showing CGRP immunoreactivity in an inflamed hind paw injected with the AP2 inhibiting peptidomimetic.

FIG. 21 shows injection of AP2α2 shRNA significantly reduces AP2α2 protein expression in ipsilateral DRGs. (Top) Whole western blot image from FIG. 7. Visible bands correspond to AP2α2. (Bottom) Whole western blot image from FIG. 7. Visible bands correspond to actin.

### DETAILED DESCRIPTION OF THE DISCLOSURE

Although claimed subject matter will be described in terms of certain embodiments/examples, other embodiments/examples, including embodiments/examples that do not provide all of the benefits and features set forth herein, are also within the scope of this disclosure. Various structural, logical, and process step changes may be made without departing from the scope of the disclosure.

Ranges of values are disclosed herein. The ranges set out a lower limit value and an upper limit value. Unless otherwise stated, the ranges include all values to the magnitude of the smallest value (either lower limit value or upper limit value) and ranges between the values of the stated range.

Throughout this application, the singular form encompasses the plural and vice versa. The references cited in this application are hereby incorporated by reference. All sections of this application, including any supplementary sections or figures, are fully a part of this application.

The term “treatment” as used herein refers to reduction in one or more symptoms or features associated with the presence of the particular condition being treated. Treatment does not necessarily mean complete cure or remission, nor does it preclude recurrence or relapses. For example, treatment in the present disclosure means reducing pain (e.g., decreasing pain sensitivity) or increasing pain sensitivity.

The term “therapeutically effective amount” as used herein refers to an amount of an agent sufficient to achieve, in a single or multiple doses, the intended purpose of treatment. Treatment does not have to lead to complete cure, although it may. Treatment can mean alleviation of one or more of the symptoms or markers of the indication. The exact amount desired or required will vary depending on the particular compound or composition used, its mode of administration, patient specifics and the like. Appropriate effective amount can be determined by one of ordinary skill in the art informed by the instant disclosure using only routine experimentation. Treatment can be orientated symptomatically, for example, to suppress symptoms. It can be effected over a short period, over a medium term, or can be a long-term treatment, such as, for example within the context of a maintenance therapy. Treatment can be continuous or intermittent.

Unless otherwise indicated, nucleic acids are written left to right in 5′ to 3′ orientation; amino acid sequences are written left to right in amino to carboxyl orientation, respectively. Numeric ranges recited within the specification are inclusive of the numbers defining the range and include each integer within the defined range. Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

The present disclosure provides agents and methods of using these agents to treat or prevent pain and induce anesthesia. The agents may be peptides, siRNAs, and/or shRNAs targeting adaptin protein 2 (AP2)-clathrin mediated endocytosis (CME). In one aspect, use of these agents will diminish or eliminate the need for narcotics (e.g., opioids) to combat pain.

The present disclosure provides peptides having a sequence according to Table 1.

TABLE 1

| Di-leucine based | | |
|---|---|---|
| peptides | Parent Protein | Sequence |
| Ap2 inhibitor peptide | CD4 | RMSEIKRLLSE (SEQ ID NO: 1) |
| P1 | EGFR | RLRTLRRLLQE (SEQ ID NO: 2) |
| P2 | Kcnt1 | RLEPNDIVYLIRS (SEQ ID NO: 3) |
| P3 | CD3 | RASDKQTLLPNQ (SEQ ID NO: 4) |
| P4 | CD3 | RASDKQTLLPNQ (SEQ ID NO: 5) |
| Scrambled sequence | CD4 | IERLSEMSLRK (SEQ ID NO: 6) |

Underline-(D/E/S/T)XXXL(L/I) AP2 binding motif shown in experiments
Bold-phosphorylated residue

The present disclosure also provides peptides comprising the sequence (D/E/S/T)XXXL(L/I) (SEQ ID NO:7). This sequence may be represented as X¹X²X³X⁴LX⁵ (SEQ ID NO:7), where X¹ is D, E, S, or T, where the D, E, S, and/or the T is optionally phosphorylated, X², X³, and X³ are independently chosen from any amino acid (e.g., canonical amino acids (e.g., X² may be I, L, or K; X³ may be K, R, V, or Q; X⁴ may be R, Y, or T) or non-canonical amino acids), and X⁵ is L or I. A peptide of the present disclosure may be 6, 7, 8, 9, 10, 11, 12, 13, 14, 15, 16, 17, 18, 19, or 20 amino acid residues long. In various examples, the peptide has the following sequence: EIKRLL (SEQ ID NO:9), TLRRLL (SEQ ID NO:10), DIVYLI (SEQ ID NO:11), or DKQTLL (SEQ ID NO:12). In various examples, the peptide is 10 to 13 amino acid residues long (e.g., 10, 11, 12, or 13). Without intending to be bound by any particular theory, it is considered that peptides having a total length of 10 to 13 amino acids may have desirable cell penetration and target binding properties. In various examples, any amino acid residue (e.g., any combination or all of the amino acid residues) of SEQ ID NO:7 may be phosphorylated

In an embodiment, the D/E/S/T in the peptide sequence is phosphorylated. In the experiments reported herein, the T was phosphorylated. By extension, the phosphorylated T could be replaced by phosphorylated S.

In an embodiment, the (D/E/S/T)XXXL(L/I) (SEQ ID NO:7) sequence is preceded by S or T ((S/T)(D/E/S/T)XXXL(L/I) (SEQ ID NO:8)), which may optionally be phosphorylated. SEQ ID NO:8 may be represented as X⁶X¹X²X³X⁴LX⁵. (SEQ ID NO:8), where X¹ is D, E, S, or T and X¹ optionally phosphorylated, X², X³, and X³ are independently chosen from any amino acid (e.g., X² may be I, L, or K; X³ may be K, R, V, or Q; X⁴ may be R, Y, or T), X⁵ is L or I, and X⁶ is S or T.

In another embodiment, the C terminus or the amino acid immediately preceding the C terminus of a peptide of the present disclosure may optionally be phosphorylated.

In a preferred embodiment, the peptide is lipidated. Moieties that may be used for lipidation include myristoyl (C₁₄), octanoyl (C₈), lauroyl (C₁₂), palmitoyl (C₁₆) and stearoyl (C₁₈).

In various embodiments, the N-terminus or N-termini of the peptide(s) is/are myristoylated. Accordingly, the N terminus of the peptide may be lipidated. Alternatively, the C terminus of the peptide may be lipidated. For example, lipidation of the C terminus may be useful when the C terminus is lysine.

Additionally, the subject disclosure describes an RNAi agent directed against AP2-CME mRNA (agent for use in RNA interference mediated silencing or downregulation of AP2-CME mRNA). RNAi agents are commonly expressed in cells as short hairpin RNAs (shRNA). shRNA is a RNA molecule that contains a sense strand, antisense strand, and a short loop sequence between the sense and antisense fragments. shRNA is exported into the cytoplasm where it is processed by dicer into short interfering RNA (siRNA). siRNA are typically 20-23 nucleotide double-stranded RNA molecules that are recognized by the RNA-induced silencing complex (RISC). Once incorporated into RISC, siRNA facilitate cleavage and degradation of targeted mRNA. Thus, the RNAi agent can be a siRNA or a shRNA. In one embodiment, the agent is a siRNA for use in RNA interference (RNAi) mediated silencing or downregulation of AP2-CME mRNA. The RNAi agent may be human, non-human or partially humanized.

shRNA can be expressed from any suitable vector such as a recombinant viral vector either as two separate, complementary RNA molecules, or as a single RNA molecule with two complementary regions. In this regard, any viral vector capable of accepting the coding sequences for the shRNA molecule(s) to be expressed can be used. Examples of suitable vectors include but are not limited to vectors derived from adenovirus, adeno-associated virus, retroviruses (e.g., lentiviruses), rhabdoviruses, murine leukemia virus, herpes virus, and the like. A preferred virus is a lentivirus. The tropism of the viral vectors can also be modified by pseudotyping the vectors with envelope proteins or other surface antigens from other viruses. As an alternative to expression of shRNA in cells from a recombinant vector, chemically stabilized shRNA or siRNAs may also be used administered as the agent in the method of the present disclosure. Vectors for expressing shRNA which in turn produces siRNA once introduced into a cell are commercially available. Further, shRNAs or siRNAs targeted to virtually every known human gene are also known and are commercially available.

The present disclosure also provides a pharmaceutical composition comprising a pharmaceutically acceptable carrier and said peptide and/or said RNAi agent directed against AP2-CME mRNA and, optionally, an analgesic agent (e.g., nonsteroidal anti-inflammatory drug (NSAID)) and/or an anesthetic agent and/or an anti-inflammatory agent (e.g., glucorticoid). Examples of analgesics include, but are not limited to, acetaminophen, aspirin, ibuprofen, naproxen, meloxicam, ketorolac, diclofenac, ketoprofen, piroxicam, and metamizole. Examples of anesthetic agents include, but are not limited to, bupivacaine, etidocaine, levobupivacaine, lidocaine, mepivacaine, prilocaine, ropivacaine, procaine, chloroprocaine, hydrocortisone, triamcinolone, methylprednisolone. Using techniques and carriers known to those of skill in the art (e.g., Remington: The Science and Practice of Pharmacy (2005) 21st Edition, Philadelphia, PA. Lippincott Williams & Wilkins), the compositions can be formulated as, for example, intramuscular, intravenous, intraarterial, intradermal, intrathecal, subcutaneous, intraperitoneal, intrapulmonary, intranasal and intracranial injections or compositions. They can also be formulated as, for example, oral, buccal, or sublingual compositions, suppositories, topical creams, or transdermal patches.

Non-limiting examples of compositions include solutions, suspensions, emulsions, solid injectable compositions that are dissolved or suspended in a solvent before use, and the like. The injections may be prepared by dissolving, suspending, or emulsifying one or more of the active ingredients in a diluent. Examples of diluents, include, but are not limited to distilled water for injection, physiological saline, vegetable oil, alcohol, and a combination thereof. Further, the injections may contain stabilizers, solubilizers, suspending agents, emulsifiers, soothing agents, buffers, preservatives, and the like. The injections may be sterilized in the final formulation step or prepared by sterile procedure. The composition of the present disclosure may also be formulated into a sterile solid preparation, for example, by freeze-drying, and can be used after sterilized or dissolved in sterile injectable water or other sterile diluent(s) immediately before use.

In an aspect, the present disclosure provides a method of treating or preventing pain or inducing anesthesia by administering a therapeutically, preventatively or anesthetically effective amount of said peptide and/or said RNAi agent directed against AP2-CME mRNA to a subject in need thereof.

In one embodiment, the subject is a human or non-human mammal.

In a further embodiment, the subject does not take opioids, does not tolerate opioids well, suffers from opioid addiction, or is at risk of relapse for opioid addiction. Opioid tolerance, addiction, or relapse risk may be determined subjectively or objectively by the subject and/or a medical professional such as a doctor or other clinician.

In one embodiment, the pain is nociceptive. In another embodiment, the pain is neuropathic. The pain may be a symptom of any disease, condition, or occurrence, such as injury (e.g., spinal cord injury, nerve injury, somatic injury or burns), chronic disease (e.g., diabetes, Herpes zoster, major depressive disorder, fibromyalgia, migraine, arthritis, cancer, multiple sclerosis, inflammatory bowel disease or HIV/AIDS), radiculopathy, chronic inflammation (e.g., chronic inflammation associated with repetitive stress, such as, for example, carpal tunnel syndrome), chemotherapy, radiation, Morton's neuroma, mechanical/thermal stress, allodynia/hyperalgesia (each of which may be mechanical, thermal or movement-associated). In an embodiment, the hyperalgesia is opioid-induced. The pain may also be post-surgical pain. The pain that is prevented may be anticipated pain, such as pain during surgery, laparoscopy, chemotherapy, dental work, radiation, and childbirth. The pain may be chronic and/or acute pain.

Chronic pain is any pain lasting for more than around 12 weeks. In another embodiment, chronic pain is pain that extends beyond the expected period of healing.

Acute pain is sharp, and does not typically last longer than around six months. Acute pain goes away when there is no longer an underlying cause of pain. Causes for acute pain include, but are not limited to, surgery, laparoscopy, broken bones, dental work, burns, cuts, labor/childbirth, and combinations thereof.

Treatment or prevention of pain can be determined, e.g., by description from the subject based on pain assessments using a variety of validated pain measurement tools (e.g., visual analog pain scale (VAS), numeric rating pain (NRS), categorical verbal rating pain scale (VRS), multidimensional scales assessing the sensory components and also cognitive and psychological dimensions of pain, health-related quality-of-life assessment, pain-related functional assessments). Non-limiting examples of pain measurement tools include the VAS, NRS, VRS, the McGill Pain Questionnaire (MPQ) and its Short Form, The Brief Pain Inventory (BPI), Neuropathic Pain Score (NPS), The Pain Self-Efficacy Questionnaire, Patient Global Impression of Change scale, The European Quality of Life Instrument (EQ 5D), Pain Disability Index (PDI), The Oswestry Disability Index (ODI), the Beck Depression Inventory and Profile of Mood States, the Wong-Baker faces pain scale, the FLACC scale (face, legs, activity, cry, and consolability), the CRIES scale (crying, requires O2 for SaO2 <95%, increased vital signs (BP and HR), expression, sleepless), the COMFORT scale, Mankoski pain scale, descriptor differential scale of pain intensity, and combinations thereof.

Pain is treated or prevented when it is at least partially ameliorated. Likewise, the method does not require complete anesthesia. For example, the treatment or prevention is considered anesthetically effective when the subject's mechanical/tactile sensitivity is at least partially decreased. The subject's mechanical/tactile sensitivity may be determined subjectively or objectively by the subject and/or a medical professional such as a surgeon, other doctor or other clinician. The anesthesia may be local or central.

A subject's pain may be ameliorated when the subject's pain (e.g., pain sensitivity) decreased. In example, a subject's pain is ameliorated when the subject's pain (e.g., pain sensitivity) is at a desired level (e.g., the pain is not uncomfortable).

In an embodiment, following the administration, the subject's pain is ameliorated/treated/prevented for 0.25-120 hours (e.g., 24-120 hours, 1-48 hours, 12-48 hours, or 24-48 hours), including all integers and decimals to the 100th place and all ranges therebetween. In another embodiment, following the administration, anesthesia is induced for 0.25-100 hours, including all integers and decimals to the 100th place and all ranges therebetween.

The peptide and/or said RNAi agent directed against AP2-CME mRNA may be administered or used alone or in combination with an analgesic and/or anesthetic and/or an anti-inflammatory agent. Examples of analgesic, anesthetic, and anti-inflammatory agents are provided above. When administered in combination, the administration or use may occur simultaneously or sequentially (in any order). Any of the foregoing may be formulated in combined formulation or in separate formulations.

Any or all of the aforementioned administration(s) may be, for example, intramuscular, intravenous, intraarterial, intradermal, intrathecal, intraperitoneal, intrapulmonary, intranasal, intracranial, oral, buccal, sublingual, subcutaneous, anal, topical, transdermal, or by nerve injection. In an embodiment, said administration is conducted by needleless injection(s).

In a preferred embodiment, shRNAs are administered directly into nerve(s).

In one embodiment, said peptide and/or said RNAi agent directed against AP2-CME mRNA is administered during a surgical procedure or labor/childbirth.

In an aspect, the present disclosure further provides kits. Kits may comprise a pharmaceutical composition comprising a peptide and/or said RNAi agent directed against AP2-CME mRNA.

In an embodiment, the kit comprises a package (e.g., a closed or sealed package) that contains a pharmaceutical composition, such as, for example, one or more closed or sealed vials, bottles, blister (bubble) packs, or any other suitable packaging for the sale, distribution, or use of the pharmaceutical compositions.

In an embodiment, the kit further comprises printed material. The printed material includes, but is not limited to, printed information. The printed information may be, e.g. provided on a label, or on a paper insert or printed on the packaging material itself. The printed information may include information that, for example, identifies the composition in the package, the amounts and types of other active and/or inactive ingredients, and instructions for taking the composition, such as, for example, the number of doses to take over a given period of time and/or information directed to a pharmacist and/or a health care provider (such as a physician) or a patient. In an example, the product includes a label describing the contents of the container and providing indications and/or instructions regarding use of the contents of the container.

The steps of the method described in the various embodiments and examples disclosed herein are sufficient to carry out the methods of the present disclosure. Thus, in an embodiment, the method consists essentially of a combination of the steps of the methods disclosed herein. In another embodiment, the method consists of such steps.

In the following Statements, various examples of the peptides, compositions, and methods of using the peptides and compositions of the present disclosure are described.

Statement 1. A peptide comprising the following sequence: X¹X²X³X⁴LX⁵ (SEQ ID NO:7) where X¹ is chosen from D, E, S, and T; X², X³, and X⁴ are independently chosen from any amino acid; and X⁵ is chosen from L and I; and where L, X¹, and/or X⁵ is optionally phosphorylated and the peptide is 6-20 amino acid residues (e.g., 6, 7, 8, 9, 10, 11, 12, 13, 14, 15, 16, 17, 18, 19, or 20) (e.g., in 10-13 amino acid residues (e.g., 10, 11, 12, or 13)) long.

Statement 2. A peptide according to Statement 1, where the C-terminal amino acid residue or the amino acid residue immediately preceding the C-terminal amino acid is phosphorylated.

Statement 3. A peptide according to Statements 1 or 2, where the peptide is lipidated.

Statement 4. A peptide according to Statement 3, wherein the lipidation is at the N-terminal amino acid residue.

Statement 5. A peptide according to Statements 3 or 4, where the lipidation is myristoylation, octanoylation, lauroylation, palmitoylation, or stearoylation.

Statement 6. A peptide according to any one of the preceding Statements, where the peptide has the following sequence: X⁶X¹X²X³X⁴LX⁵ (SEQ ID NO:8), where X⁶ is chosen from S and T, and X⁶ is optionally phosphorylated.

Statement 7. A peptide according to any one of the preceding Statements, comprising a sequence chosen from SEQ ID NOs:1, 2, 3, 4, 5, 8, 9, 10, 11, and 12.

Statement 8. A composition comprising one or more peptide according to any one of the preceding Statements and a pharmaceutically acceptable carrier.

Statement 9. A composition according to Statement 8, further comprising one or more analgesic agent and/or one or more anesthetic agent.

Statement 10. A composition according to Statements 8 or 9, wherein the one or more analgesic and/or the one or more anesthetic agent is acetaminophen, aspirin, ibuprofen, naproxen, meloxicam, ketorolac, diclofenac, ketoprofen, piroxicam, metamizole, bupivacaine, etidocaine, levobupivacaine, lidocaine, mepivacaine, prilocaine, ropivacaine, procaine, chloroprocaine, hydrocortisone, triamcinolone, methylprednisolone or a combination thereof.

Statement 11. A composition according to any one of Statements 8-10, further comprising AP2-CME targeting shRNA and/or AP2-CME targeting siRNA.

Statement 12. A method of treating pain or increasing pain sensitivity in a subject in need of treatment comprising: administering to the subject in need of treatment a therapeutically effective amount of one or more composition according to any one of Statements 8-10, wherein pain of the subject in need of treatment is ameliorated or the pain sensitivity of the subject in need of treatment is increased.

Statement 13. A method according to Statement 12, further comprising administering one or more analgesic agent and/or one or more anesthetic agent.

Statement 14. A method according to Statements 12 or 13, where the administration step is performed in anticipation of pain.

Statement 15. A method according to any one of Statements 12-14, where the subject in need of treatment has an injury, a chronic disease, a chronic inflammation, Morton's neuroma, operative/post-operative pain or a combination thereof.

Statement 16. A method according to Statement 15, where the injury is a spinal cord injury, a nerve injury, a burn, or a combination thereof.

Statement 17. A method according to Statement 16, where the chronic disease is diabetes, Herpes zoster, major depressive disorder, fibromyalgia, migraine, arthritis, amyotrophic lateral sclerosis, multiple sclerosis, inflammatory bowel disease, schizophrenia, autism spectrum disorders, cancer, radiculopathy or a combination thereof.

Statement 18. A method according to any one of Statements 12-17, where the peptide administered to the subject has a sequence chosen from SEQ ID NOs:1, 2, 3, 4, 5, 8, 9, 10, 11, 12, and combinations thereof.

Statement 19. A method according to any one of Statements 12-18, where the subject's pain is ameliorated for 1-120 hours following a single administration step.

Statement 20. A method according to any one of Statements 12-19, where the subject's pain is ameliorated for 24-120 hours following a single administration step.

The following examples are presented to illustrate the present disclosure. They are not intended to be limiting in any matter.

### Example 1

This example provides a description of methods of the present disclosure.

An in vivo DRG neuron gene knockdown technique was used to corroborate in vitro data from a previous study. Understanding how AP2-CME might impact behavioral processes in in vivo studies is difficult. Transgenic-based approaches are limited due to the essential role AP2-CME plays in developmental processes. To overcome this limitation, a spinal nerve injection technique was utilized to unilaterally transfect shRNAs targeted against the alpha-2 subunit (AP2A2) of the AP2 complex in vivo into the sciatic nerve of naïve mice. In acute and chronic inflammatory pain models, AP2A2 deficiency resulted in significant reductions in pain-like behaviors. Specifically, in the formalin assay, AP2A2-deficient mice exhibited an amelioration of pain-like behaviors attributed to peripheral nociceptor sensitization. During Complete Freund's Adjuvant (CFA) mediated chronic pain, AP2A2-deficient mice exhibited a significant increase in paw withdrawal latency during thermal behavioral testing, suggesting that AP2-CME is required for the initiation of chronic pain states. Furthermore, during established CFA chronic pain, knockdown of the AP2A2 subunit rapidly reversed thermal hyperalgesia, suggesting that AP2-CME is required for maintenance of chronic pain states.

Finally, local pharmacological inhibition of AP2-CME was used to complement genetic studies and similarly found an attenuation in acute and chronic thermal pain behaviors. Herein, a specific function for dorsal root ganglion AP2-CME in pain signaling is described and a peripheral nerve terminals as pharmacological targets for pain management were identified.

Inhibition of endocytosis, by both genetic and pharmacological approaches, resulted in robust decreases in pain-like behaviors in mice, which was surprising. Without being bound by any theory, it is believed that inhibition of endocytosis induces a membrane “suspension” that prevents internalization of membrane proteins such as Slack K_{Na} channels in DRGs during inflammation. Preventing Slack channel internalization for example would immobilize these channels at the membrane prior to inflammation, maintain basal membrane excitability and prevent inflammation-induced nociceptor hyperexcitability.

Nonetheless, it be cannot ruled out the possibility of other immobilized membrane proteins contributing to reduced pain behavior. For example, DRG neurons express both pro- and anti-nociceptive G-protein coupled receptors (GPCRs). It is possible that the inability to desensitize anti-nociceptive GPCRs may be contributing to the observed effects. It is equally possible that non-desensitizing pro-nociceptive GPCRs would exacerbate pain. Indeed, studies have shown that, formalin phase II inflammatory pain was exacerbated in beta2-arrestin knockout mice. Without being bound by any theory, it is considered that the possible net effect of suspended GPCR endocytosis on pain signaling would be minimal and that membrane ion channels controlling excitability would be more pertinent in this process.

Using both genetic and pharmacological approaches, these results revealed that the initiation of inflammatory pain states was dependent upon neuronal endocytosis. There is extensive literature on the transition from PKA signaling to PKC signaling during chronic inflammatory pain states. It was surprising that endocytosis of Slack K_{Na} channels was important in maintaining chronic inflammatory pain as prior work has shown that PKC activation causes Slack channel potentiation when heterologously expressed in CHO cells. It was observed, however, that during heterologous co-expression of Slack channels and Ywhaz, PKC activation caused the endocytosis of Slack channels and downregulation of Slack K_{Na} currents. These observations are more consistent with the idea that DRG neuronal endocytosis is important for maintaining chronic inflammatory pain states.

Furthermore, the closely related Slick channel, also contains an AP2 endocytotic dileucine motif. Prior work showed that overexpressing fast activated Slick channels in DRG neurons resulted in the inability of neurons to fire action potentials during suprathreshold stimulation. Furthermore, it was shown that Slick channels localized to large dense core vesicles containing CGRP. Without being bound by any theory, it is possible that Slick channels accumulate to the DRG neuronal membrane during inflammatory signaling and the inability to internalize them contributes to a reduction in pain behavior, especially thermal hyperalgesia. Slick channels are exclusively expressed in CGRP positive neurons, which encode heat detection.

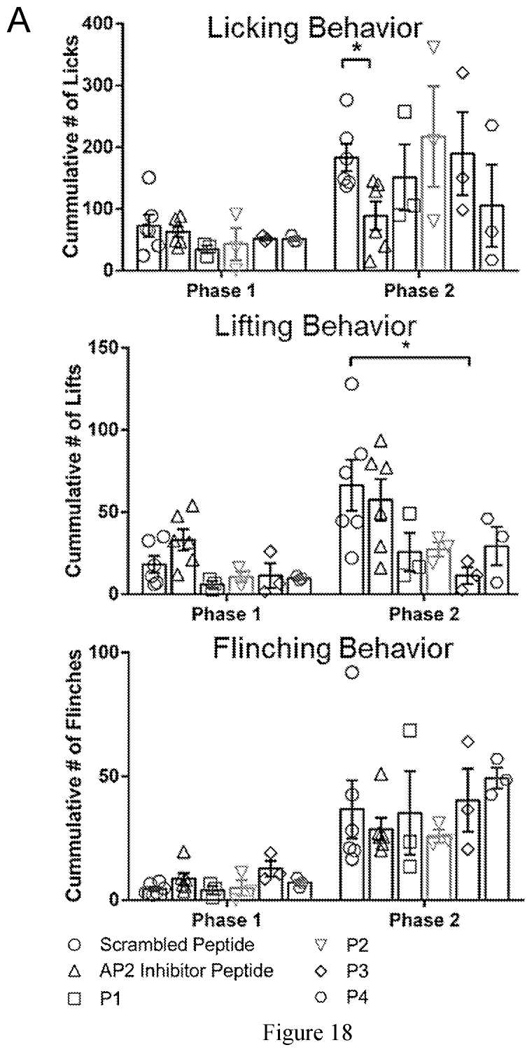

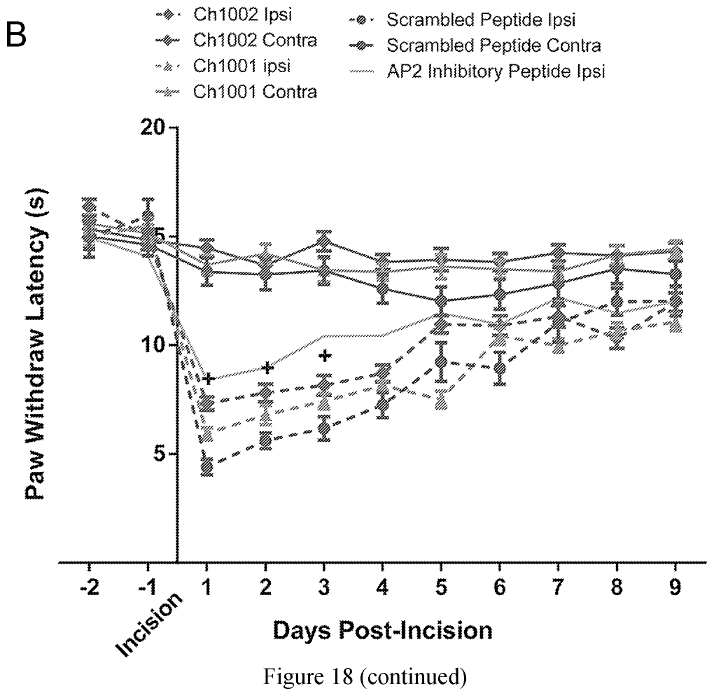

After genetically targeting AP2A2, pharmacological inhibition of endocytosis was pursued using myristoylated cell-penetrating peptides. Without being bound by any theory, it is considered that AP2-CME is important in inflammatory pain initiation and maintenance. The role of DRG peripheral terminal endocytosis from inflammatory pain processing from the possible central effects associated with gene manipulation approaches was also differentiated. In other words, the action of the peptides to be local was interpreted. Cell-penetrating peptides were used as small molecules for analgesia. Administration of the AP2 inhibitor peptide directly into the area of inflammation attenuated licking behavior in acute inflammatory pain models and resulted in a robust decrease in thermal hypersensitivity in animals 24 hours post injection in the CFA chronic inflammatory pain model. Without being bound by any theory, the efficacy of the peptide is attributed to its ability to laterally and longitudinally diffuse through the axon. Differential effects of various dileucine peptides was noted on licking vs. lifting behavior in acute formalin-induced pain where the phosphorylation status of the peptide appears to be a determining effect on efficacy in the respective behaviors (Table 1 and FIG. 18A). The data shows a rapid lateral diffusion through the membrane (single injection was effective 24 hours post injection). The data also demonstrates a slower longitudinal diffusion (duration of effect extends past 72 hours in the chronic pain model). Without being bound by any theory, we believe that the AP2 inhibitor peptides produced a prolonged inhibition of endocytosis that prevented AP2-CME-dependent alterations of nociceptor membrane proteins. Without being bound by any theory, we believe that this essentially locked the membrane in a state of biological stasis that prevented further progression to a pro-nociceptive state that potentiated DRG recovery. Without being bound by any theory, we believe that phosphorylation of the peptides may enhance peptide uptake and/or efficacy in inhibiting the AP2 complex.

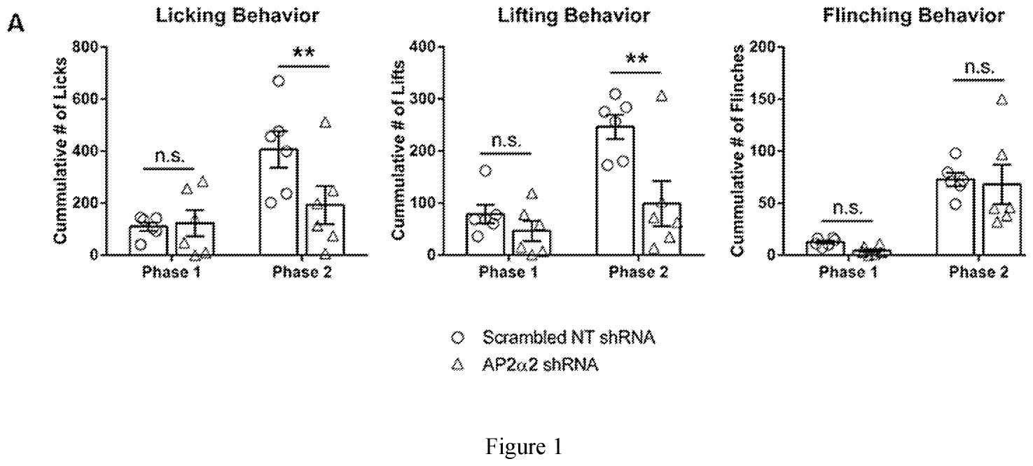

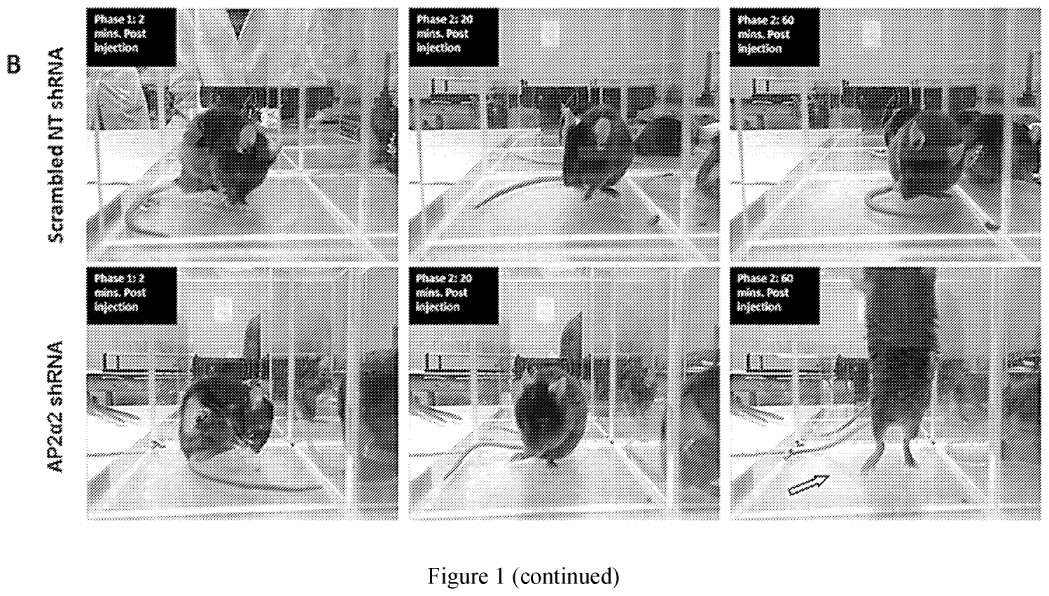

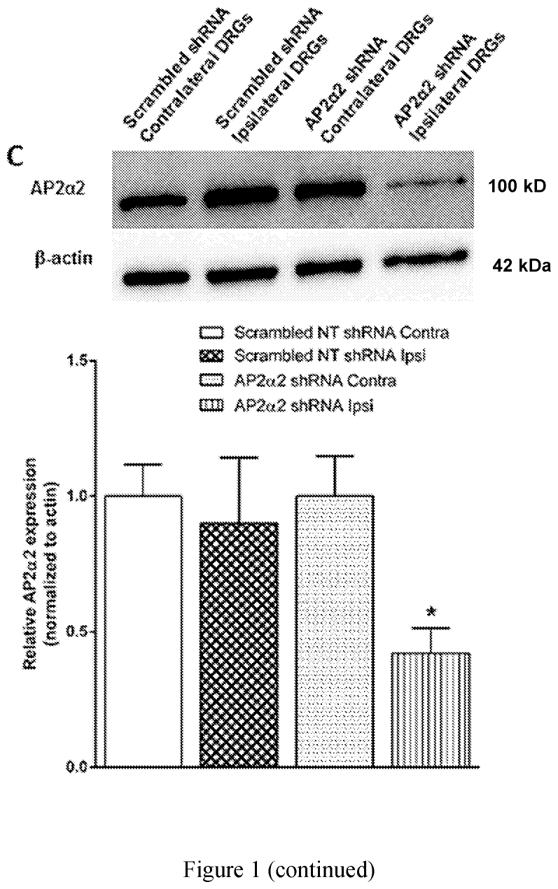

In vivo AP2A2 knockdown decreased acute inflammatory pain behavior. Previous work demonstrated that inhibiting the AP2-CME in vitro reduced PKA-induced DRG neuronal hyperexcitability and the AP2A2 subunit was shown to directly bind to Slack K_{Na} channels in DRG neurons after PKA stimulation. The consequences of in vivo knockdown of AP2A2 on pain behavior was investigated. A spinal nerve injection technique of non-viral vectors containing short hairpin RNA (shRNA) sequences was used. This technique allowed for shRNA plasmid delivery to DRG sensory neuron cell bodies via axonal retrograde transport. Intra-spinal nerve injection of AP2A2 shRNAs was conducted in naïve male and female mice, and seven days later, we assessed acute pain using the formalin assay. Intraplantar (i.pl.) injection of 5% formalin induced a biphasic inflammatory pain response associated with this acute inflammatory pain model. Briefly, the formalin assay can be divided into two phases (Phase I and Phase II). Phase I is characterized by a brief behavioral response thought to be due to direct activation of nociceptors by formalin, and phase II is a prolonged response resulting from both peripheral and central sensitization, the latter of which is due to persistent nociceptive input into the spinal cord. Knockdown of the AP2A2 subunit did not significantly alter phase I responses; however, it was noted significant reduction in phase II responses (FIG. 1A). The reduced pain phenotype was readily observable, as mice displayed diminished nocifensive responses (FIG. 1). AP2A2 silencing was confirmed by Western analysis, as mice were sacrificed after the assay to verify protein knockdown. AP2A2 protein expression was found to be significantly reduced after unilateral shRNA-dependent knockdown (FIG. 1C).

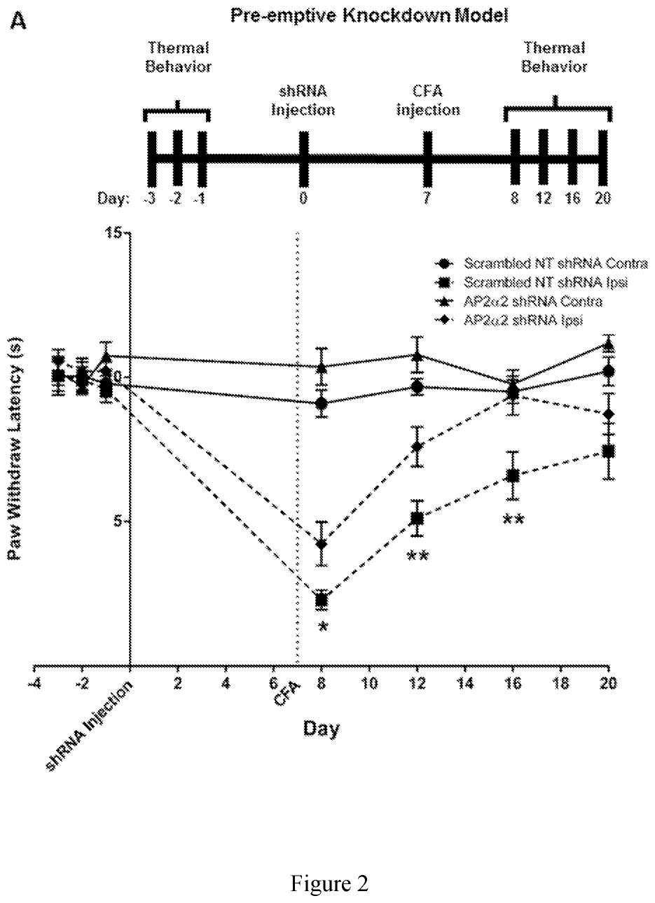

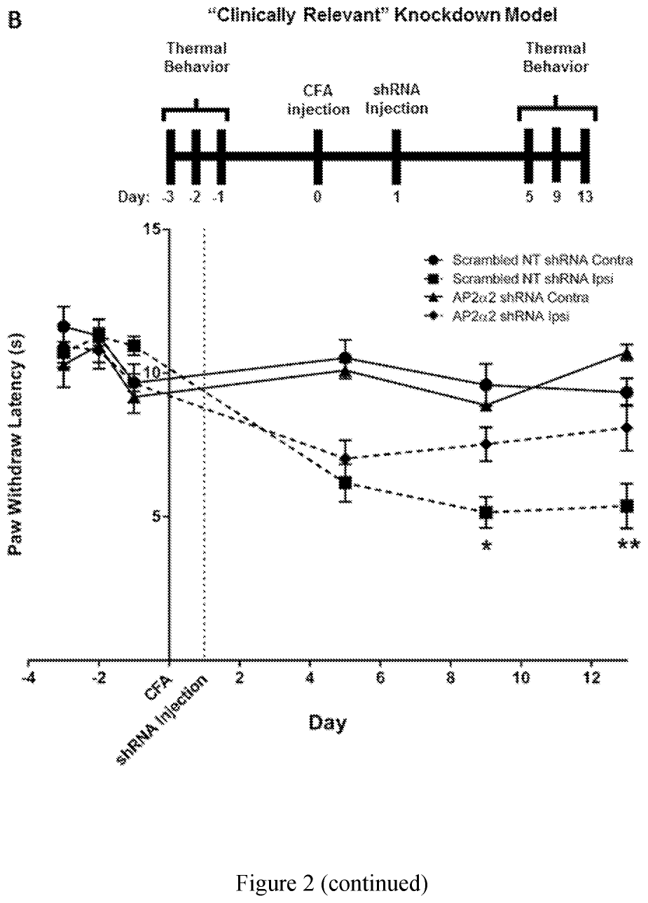

In vivo AP2A2 knockdown decreased chronic inflammatory pain behavior. When CFA is injected into the rodent hind paw, it elicits a strong immune-mediated inflammatory response that produces hypersensitivity to various innocuous stimuli-closely mimicking chronic inflammatory pain responses in humans. The consequences of AP2A2 deficiency in the development of CFA-induced inflammatory pain were investigated. The schematic representation of the experimental outline is depicted in the top of FIG. 2A. Baseline thermal responsiveness was conducted prior to intra-spinal nerve injection of AP2A2 shRNAs in naïve male and female mice. After spinal nerve surgery, animals were allowed to recover for 7 days prior to CFA injection. CFA was injected into the ipsilateral hind paw and measured thermal responsiveness. CFA ampules (Thermo Scientific) were used to ensure that each animal receives CFA with an identical activity; bolstering reproducibility of results. Mice injected with control shRNAs displayed an expected decrease in paw withdrawal latency (PWL) after CFA injection, previously observed in this strain of mice. However, AP2A2 deficiency attenuated PWL at multiple testing increments (FIG. 2A). Without being bound by any theory, these results suggest that AP2A2 is required for the development of CFA-induced hyperalgesia. It was explored whether AP2A2 knockdown could attenuate established CFA-inflammatory pain. The schematic representation of the experimental outline is depicted in the top of FIG. 2B. Again, it was established basal thermal responsiveness in ipsilateral and contralateral paws followed by CFA injection. As shown in FIG. 2A, CFA caused a peak PWL at 24 hours, followed by gradual return to baseline values, exhibiting a typical CFA response. It was found that in mice, the spinal nerve injection technique is minimally invasive, robust, quick and highly reproducible. Mice immediately exhibited exploratory behavior and climbing after recovery from the anesthesia. Thus, it was decided to conduct the surgery 24 hours after CFA was injected into the hind paw and start conducting thermal behavior testing 4 days after surgery to be able to still assess hyperalgesia behavior before recovery. It was found that knocking down AP2A2 resulted in a significant attenuation in PWL compared to control shRNA, expediting the return to baseline thermal responsiveness (FIG. 2B). These results suggest that targeting AP2-CME during established chronic inflammatory pain results in pain relief.

Cell-penetrating AP2 peptide inhibitors reduced acute and chronic inflammatory pain behaviors. Although AP2A2 was shown to be expressed extra-synaptically, unlike the presynaptic isoform AP2A1, AP2A2 knockdown was not affecting synaptic transmission in the spinal cord was investigated. The absence of significant reduction in Phase I formalin behavior (FIG. 1A) suggested that synaptic transmission was unchanged by genetic manipulation. Despite this, a cell-penetrating AP2 inhibitor was used locally to modulate peripheral nerve ending function.

Myristoylated peptides have been used to target nerve ending function in vivo. Specifically, the AP2 inhibitors are dileucine based peptides. Dileucine based peptides have been shown structurally to bind to the 62 interface of the AP2 complex. Moreover, it was previously shown that the AP2 inhibitory peptide blocks clathrin recruitment to the membrane, blocks Slack channel internalization in primary DRG neurons and prevents hyperexcitability during PKA stimulation.

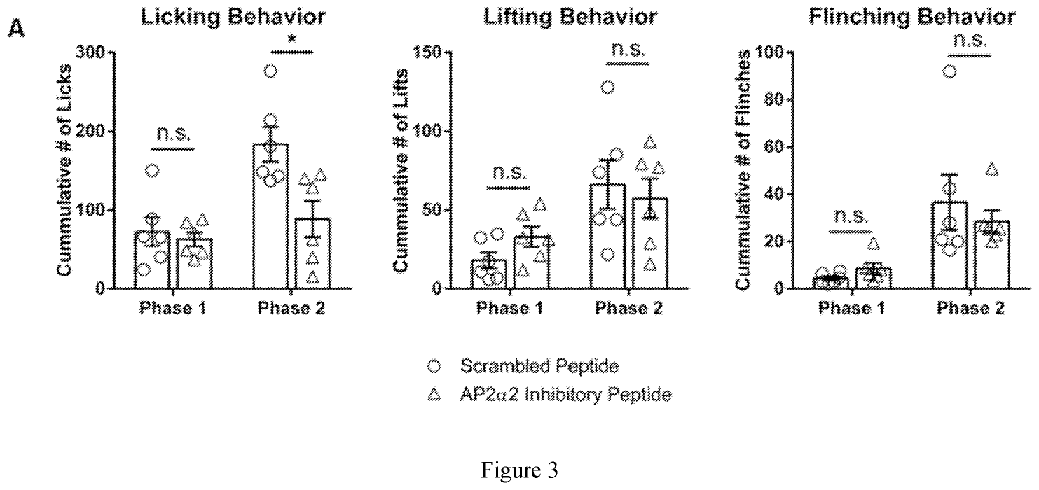

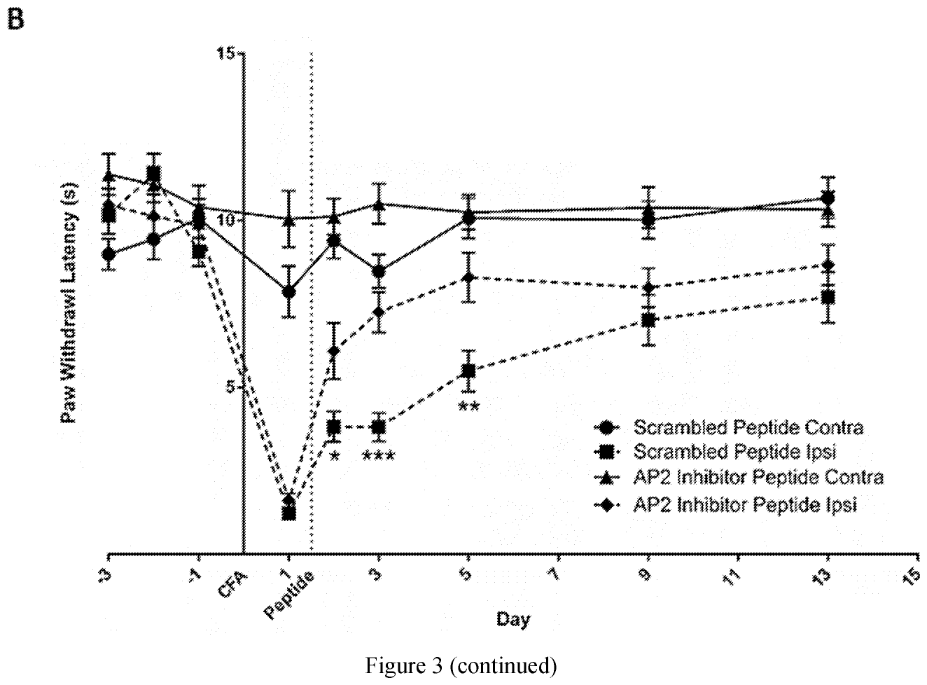

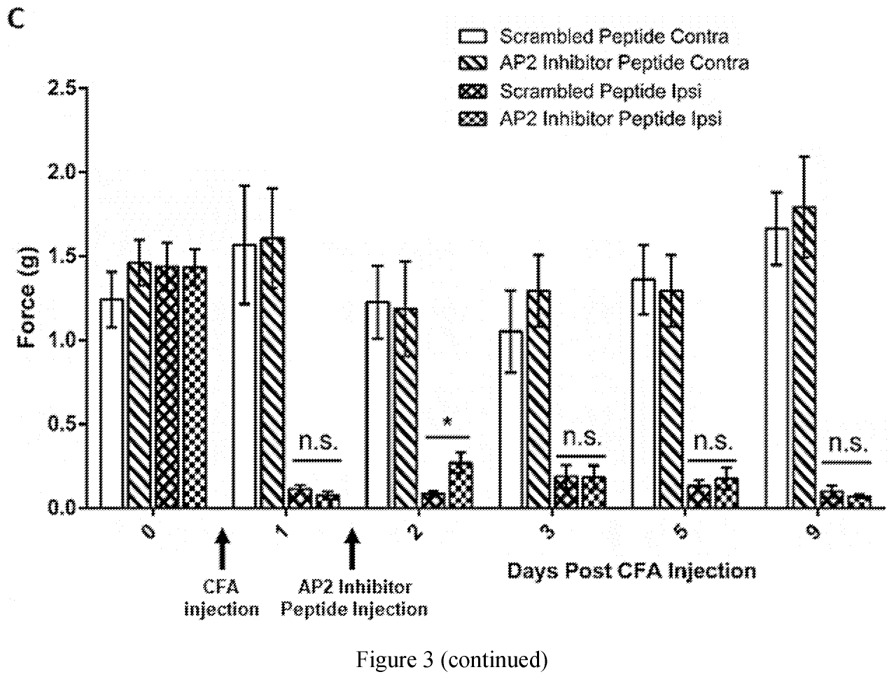

Mice were given a single i.pl. injection of either the AP2 inhibitor peptide or a scrambled peptide (100 μM, 20 l) to the right hind paw, 24 hours before injection with 5% formalin into the same paw. The peptide sequences are set forth in Table 1. Pretreatment with the AP2 inhibitor peptide significantly reduced Phase II paw licking pain-like behavior compared to the scrambled peptide (FIG. 3A). Reduction Phase II pain behavior was observed when examining a series of dileucine based peptides (Table 1 and FIG. 18A). This indicates that AP2-CME can be locally inhibited in vivo using these dileucine-based peptides.

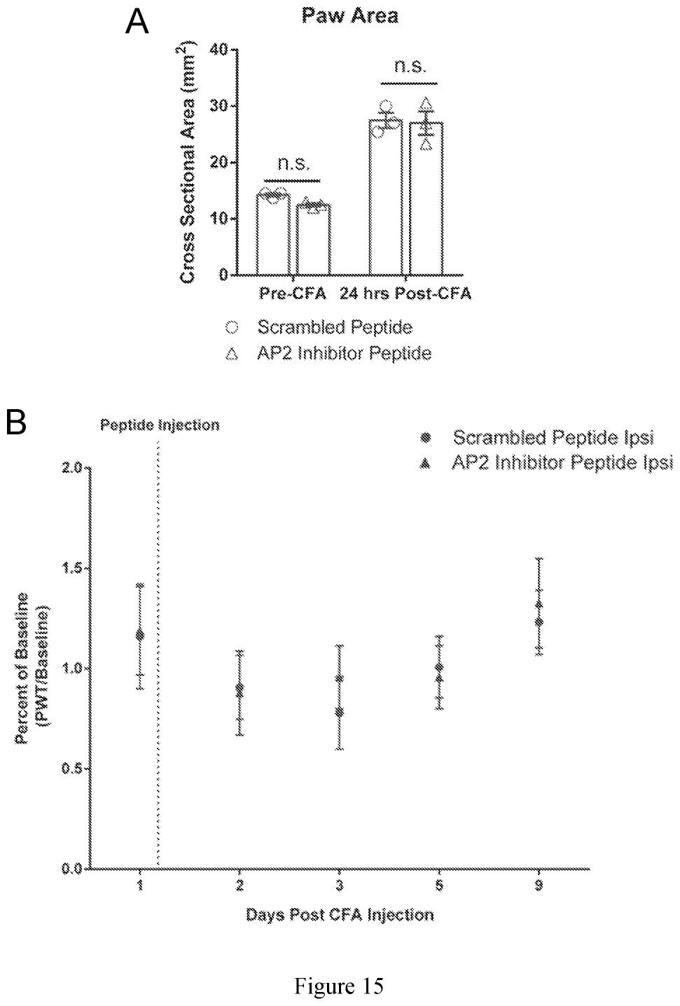

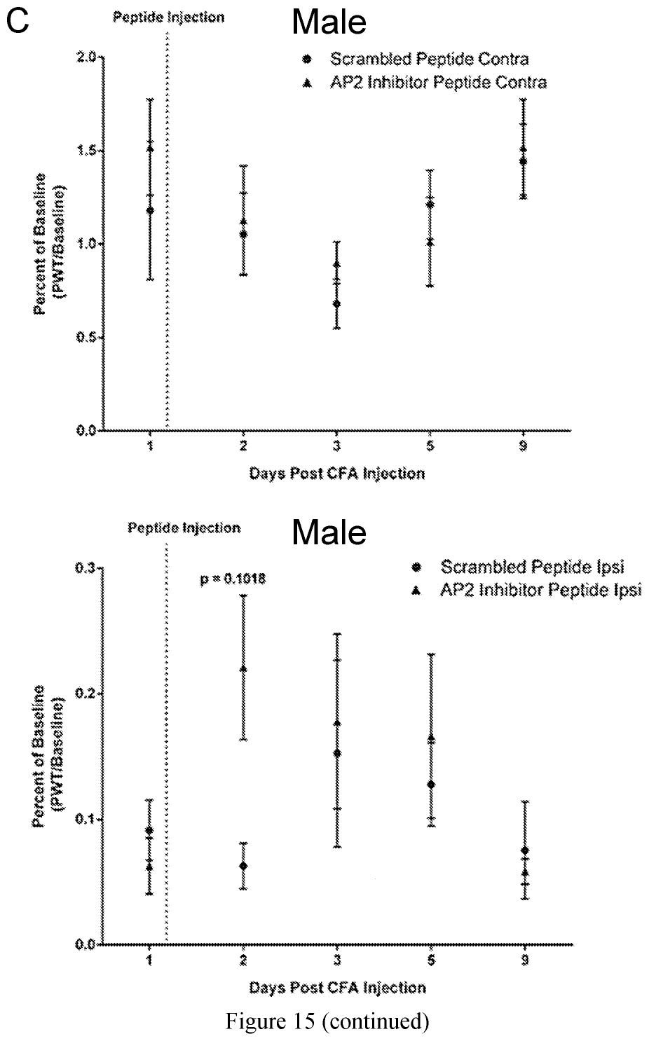

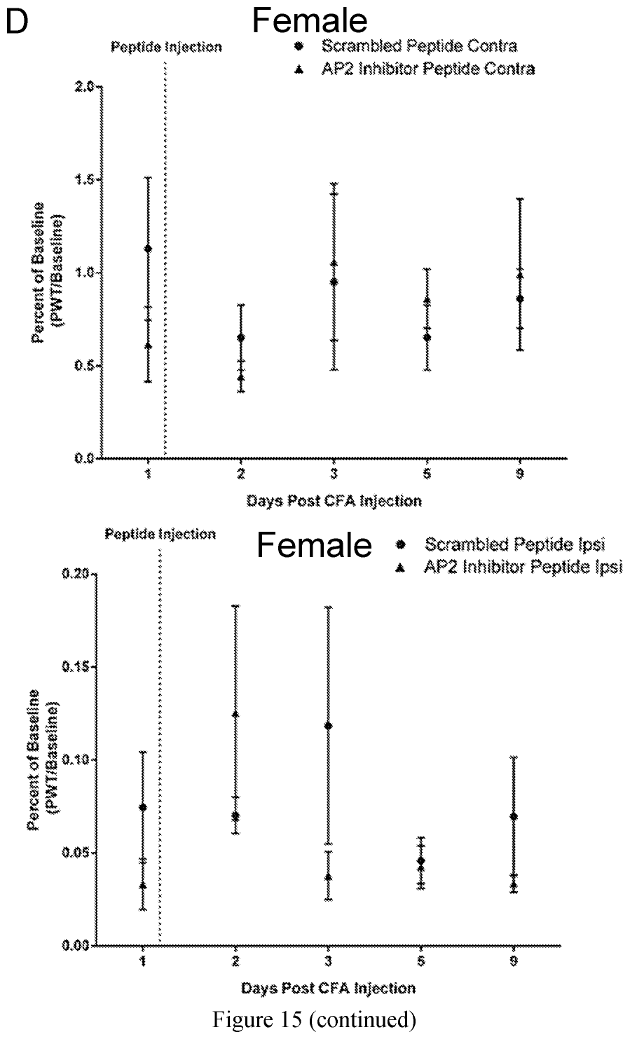

One limitation of the formalin assay and employing this local peptide approach is that afferents at the site of formalin injection receive the highest concentrations of formalin, are most likely to undergo fixation. These same afferents would also receive the highest concentration of peptide, thus the formalin assay potentially underestimated the true analgesic potential of these peptides. Thus, it was determined that the analgesic properties during established CFA-induced chronic pain. In this case, the AP2 peptide inhibitor and the scrambled peptide control were directly injected into an inflamed paw 24 hours after CFA injection. Afterwards, thermal responsiveness following a single local administration of AP2 peptide was assessed and compared to the scrambled peptide control. Within one day of the AP2 inhibitor administration, a significant attenuation of thermal hyperalgesia was observed. Moreover, the reduction in thermal hyperalgesia persisted for 96 hours again, after a one-time injection (FIG. 3B). Mechanical allodynia was assessed using the von Frey test and found that one-time administration of the AP2 peptide inhibitor produced a small yet significant reduction in pain behavior 24 hours after administration. However, the effect was modest and not long-lasting (FIG. 3C). These data suggest the AP2 inhibition and reduction in pain behavior showed selectivity for thermal hyperalgesia over mechanical allodynia. It was also noted that neither genetic knockdown nor peptide inhibition affected inflammatory edema (FIG. 15A). This suggested that the pain behavior effects were attributable to neuronal AP2-CME inhibition, not inhibition of inflammatory cell endocytosis.

### Experimental Materials and Methods

Animals. C57BL/6 mice were purchased from Envigo. All animals used were housed in the Laboratory Animal Facilities located at the University at Buffalo Jacobs School of Medicine and Biomedical Sciences on a 12-hour light/dark cycle. Male C57BL/6 mice were single housed due to aggression issues, females were grouped housed 4 per cage. All animals were given access to food and water ad libitum. All animal experimentation was conducted in accordance with the guidelines set by the “Guide for the Care and Use of Laboratory Animals” provided by the National Institute of Health. All animal protocols were reviewed and approved by the UB Institute Animal Care Use Committee.

In-Vivo Transfection with JetPEI®. Nerve injection was conducted as previously described. Briefly, C57BL/6 mice were anesthetized (induction: 3%, maintenance: 2%) and placed in a prone position. After the animals were under a surgical plane of anesthesia, denoted by a loss of reaction to both a tail and hind paw pinch, the dorsal area of the ipsilateral hind limb was shaved, against the grain, from the lumbar spinal area to just above the patella. The area was then disinfected using chlorohexidine, followed by a swab of ethanol, and finally a few drops of iodine. After disinfection, a 3 cm posterior longitudinal incision is made at the lumbar segment of the spine. Utilizing sterile toothpicks, ipsilateral paraspinal muscle was carefully separated near the L4 vertebrae to expose the sciatic nerve. The nerve was then manipulated slightly to ease injection. 1.5 μl of PEI/shRNA plasmid DNA polyplexes at an N/P ratio of 8 were injected directly in the spinal nerve of the right hind paw slowly using a syringe connected to a 32-gauge needle (Hamilton 80030, Hamilton, Reno, NV). AP2α2 shRNAs and control shRNA were purchased from Santa Cruz Biotechnology (Santa Cruz, CA, USA). Following injection, the needle was maintained in the sciatic nerve for at least 1 min to promote diffusion of solution and also to minimize leakage. After complete hemostasis was confirmed the wound was sutured with wound clips and mice were observed, post surgically, to ensure no adverse effects due to the injection. Mice were given 7 days of recovery before nociception testing resumed.

Cell-Penetrating Peptide Preparation. Custom myristoylated peptides were ordered from Genscript and stored in a −20° C. freezer upon arrival. Myristoylated peptides were dissolved in 500 μL of DMSO to create a working stock solution. Appropriate volumes of the DMSO stock solution was dissolved in 1 mL of sterile saline to generate 100 μM aliquots for future testing. These aliquots alongside any stock solutions were frozen at −80° C. until needed, at which point one aliquot was thawed, injected, then discarded to minimize freeze-thaw cycles of samples. In formalin-peptide experiments, animals received a 20 μL intraplantar injection of dissolved peptide 24 hours prior to experimentation. In FCA-peptide experiments, animals received a 20 μL intraplantar injection of dissolved peptide 24 hours post FCA injection.

In brief, peptides were synthesized by the solid phase synthesis method. This involved a stepwise incorporation of amino acids in vitro in a C- to N-terminal direction (opposite to the direction of protein synthesis in biological systems in vivo). Synthesis was based on the formation of a peptide bond between two amino acids in which the carboxyl group of one amino acid is coupled to the amino group of another amino acid. This process was repeated until the desired peptide sequence was obtained. The side chains of all amino acids were capped with specific “permanent” groups that could withstand continuous chemical treatment throughout the cyclical phases of synthesis and cleaved just prior to the purification of nascent peptide chain. Additionally, the N-terminal of each incoming amino acid was protected with 9-fluorenylmethoxycarbonyl (Fmoc) groups, which were removed by a mild base in each cycle to allow for the incorporation of the next amino acid to the chain. These Fmoc groups prevented non-specific reactions during synthesis that would have led to changes in length or branching of the peptide chain. Deprotection usually results in the production of cations with the potential to alkylate the functional groups on the peptide chain. Therefore, scavengers such as water, anisol or thiol derivatives were added during deprotection on to block free reactive species. Myristoylation was achieved by N-myristoyltransferase, the enzyme that catalyzes protein N-myristoylation (at the N-terminus). For peptides containing one or more of these hydroxy-amino acids, selective phosphorylation can be achieved by orthogonal protection or by Fmoc-protected phosphorylated amino acids.

RNAi. shRNA can be expressed from any suitable vector such as a recombinant viral vector either as two separate, complementary RNA molecules, or as a single RNA molecule with two complementary regions. In this regard, any viral vector capable of accepting the coding sequences for the shRNA molecule(s) to be expressed can be used. Examples of suitable vectors include but are not limited to vectors derived from adenovirus, adeno-associated virus, retroviruses (e.g., lentiviruses), rhabdoviruses, murine leukemia virus, herpes virus, and the like. A preferred virus is a lentivirus. The tropism of the viral vectors can also be modified by pseudotyping the vectors with envelope proteins or other surface antigens from other viruses. As an alternative to expression of shRNA in cells from a recombinant vector, chemically stabilized shRNA or siRNAs may be used. Vectors for expressing shRNA (which produce siRNA once introduced into a cell) are commercially available.

Formalin Assay. Male and female C57BL/6 mice were randomly assigned to either control or experimental groups. Animals were habituated to the formalin testing chamber for 30 minutes or until exploratory behavior ceased the day of experimentation. Following the habituation period, animals were removed from the chamber and given an intraplantar injection of 5% formalin into the ipsilateral hind paw, then immediately placed back into the testing chamber and recorded. Animals were recorded for at least 90 minutes after formalin injection using Active WebCam software. Videos were subsequently scored for number of paw licks, number of paw lifts, and number of full body flinches. All behaviors were scored for a full minute, every five minutes, for 90 minutes of video recording.

Freund's Complete Adjuvant Chronic Pain Model. C57BL/6 mice were anesthetized (induction: 3%, maintenance: 2%) and placed in a prone position. After the animals were under a surgical plane of anesthesia, denoted by a loss of reaction to both a tail and hind paw pinch, they received a 20 μL injection of Imject™ Freund's Complete Adjuvant (FCA; Thermo Fisher Scientific) and allowed to recover. Behavior testing resumed 24 hours post FCA injection. Each cohort of animals received FCA from previously unopened, vacuum sealed glass ampules to minimize variations between groups.

Hargreaves Assay. Animals were placed on an enclosed elevated frosted glass platform (Ugo Baseline) and allowed 30 minutes for habituation. Once exploratory behavior ceased, an automatic Hargreaves apparatus was maneuvered (Ugo Baseline) underneath the hind paw(s) of the animals. Paw withdrawal latency was calculated as the average of four trials per hind limb. Each trial was followed by a 5-minute latency period to allow adequate recovery time between trials.

Von frey assay. Animals were placed on an enclosed elevated wire-mesh platform (Ugo Baseline) and allowed 30 minutes to habituate to their enclosure. Afterwards, Touch Test Sensory Probes (Stoelting) were applied to the plantar surface of the contralateral and ipsilateral hind paw. Filaments were applied in ascending order, with a 5-minute latency in-between filament presentations, following the Simplified Up-Down method (SUDO) for mechanical nociception testing. In short, the middle filament, of the series, was presented to the hind paw of the animal. If a response was elicited, the next filament to be presented would be the next lowest filament in the series. If no response was elicited, the next filament to be presented would be the next highest filament in the series. This method of filament presentation was repeated 5 times, with the 5th filament presentation being the last one. Then an adjustment factor was added to the filament value and the force of paw withdrawal was calculated utilizing a series of conversion equations.

Western Blot Analysis. Total protein was collected from dorsal root ganglion (DRG) tissue collected from animals following experimentation. DRGs were homogenized in chilled RIPA buffer containing a protease inhibitor (Sigma) and stored at −80° C. until needed. All samples were run on Mini-PROTEAN TGX Precast Gel (Bio-Rad) and transferred to a 0.45 m nitrocellulose membrane (BioRad). Membranes were probed overnight at 4° C. with rabbit anti-AP2α2 (1:1000, Abcam) and rabbit anti-3-Actin (1:1000, Sigma) in 5% bovine serum albumin (BSA) prepared in 1× tris-buffered saline-tween (TBST). On the following day, membranes were washed three times for five minutes in 1×TBST before being incubated for 1 hour at room temperature in a secondary anti-rabbit horseradish peroxidase conjugate antibody (1:5000; Promega) prepared in a 5% BSA in 1×TBST solution. After secondary anti-body incubation, the membrane was washed more three times for five minutes per wash before being developed and imaged. Bands were visualized with enhanced chemiluminescence on a Chemidoc Touch Imaging System (Bio-rad) and quantified with Image J Software (NIH). Each experiment was repeated at least three times.

Statistical Methods. All statistical tests were performed using Prism (GraphPad). The data are shown as means±s.e.m. Power analysis was conducted for animal experiments to achieve detection limits with an α-value set at 0.05. Statistical significance was determined utilizing a p-value<0.05 for all experiments. Two-way ANOVA statistical tests with multiple comparisons and Bonferroni post hoc correction were used for all statistical analyses unless otherwise stated.

### Example 2

This example provides a description of methods of the present disclosure.

Nociceptor endocytosis were locally disrupted and various inflammatory pain models were used to characterize the in vivo contribution of extra-synaptic AP2-CME to inflammatory pain. Provided is further evidence for peptidergic nociceptors as executive regulators of inflammatory pain. The present disclosure highlights the ability of lipidated peptidomimetics to target superficial nerve afferents and to provide long-lasting analgesia. Additionally, described is the sexually dimorphic differences in pain behavior during inflammation across pain models and animal species.

Animals: All animals were purchased from Envigo and age/weight matched for all experiments. All animals used were housed in the Laboratory Animal Facilities located at the University at Buffalo (UB) Jacobs School of Medicine and Biomedical Sciences on a 12-hour light/dark cycle. For consistency, all animals were singly housed for the duration of experiments. All animals were given access to food and water ad libitum. All animal experimentation was conducted in accordance with the guidelines set by the “Guide for the Care and Use of Laboratory Animals” provided by the National Institute of Health. All animal protocols were reviewed and approved by the UB Institute Animal Care Use Committee.

In-Vivo Transfection of Sciatic Nerves with α2 targeted shRNAs and in vivo-jetPEI®: Nerve injection was conducted as previously described. Briefly, C57BL/6 mice were anesthetized and placed in a prone position. After disinfection, a 3 cm posterior longitudinal incision is made at the lumbar segment of the spine. Utilizing sterile toothpicks, ipsilateral paraspinal muscle was carefully separated to expose the sciatic nerve. Using autoclaved sticks, the nerve was manipulated slightly to ease injection. 1.5 uL of PEI/shRNA plasmid DNA polyplexes, at an N/P ratio of 8, was injected directly into the right sciatic nerve using a syringe connected to a 32-gauge needle (Hamilton 80030, Hamilton, Reno, NV). AP2α2 shRNAs and control shRNA were purchased from Santa Cruz Biotechnology (Santa Cruz, CA, USA). Following injection, the needle was maintained in the sciatic nerve for at least 1 min to promote diffusion of the polyplexes. The wound was closed with wound clips and mice were post surgically observed to ensure no adverse effects due to the injection. Mice were given 7 days of recovery before behavioral testing resumed.

Myristoylated Peptide Preparation: Custom lipidated peptidomimetics were ordered from Genscript® and lyophilized samples were stored in a −20° C. freezer upon arrival. Sequences of peptides used in the study can be found in Table 1. Lipidated peptidomimetics were initially dissolved in 10 μL of DMSO to create a working stock solution. Appropriate volumes of the DMSO stock solution was dissolved in 1 mL of sterile saline to generate 100 μM aliquots for future testing. Final DMSO concentration was <0.05%. These aliquots alongside any stock solutions were frozen at −80° C. until needed, at which point one aliquot was thawed, injected, then discarded to minimize freeze-thaw cycles of samples.

Formalin Assay: Male and female C57BL/6 mice were randomly assigned to either control or experimental groups. Animals received a 20 μL intraplantar injection of 100 M (3.154 μg total) of the lipidated peptidomimetic 24 hours prior to experimentation. Animals were habituated to the formalin testing chamber for 30 minutes or until exploratory behavior ceased the day of experimentation. Following the habituation period, animals were removed from the chamber and given an intraplantar injection of 5% formalin into the ipsilateral hind paw, then immediately placed back into the testing chamber and recorded. Animals were recorded for at least 90 minutes after formalin injection using Active WebCam software. Videos were subsequently scored for number of paw licks, number of paw lifts, and number of full body flinches. All behaviors were scored for a full minute, every five minutes, for 90 minutes of video recording. Scorers were blinded to experimental conditions.

Complete Freund's Adjuvant Induced Inflammatory Pain: Male and female C57BL/6 mice were randomized into experimental and control groups. In order to maintain consistency in regards to site of injection, mice were anesthetized and injected with a 32-gauge disposable syringe filled with 20 μL of Imject™ Complete Freund's Adjuvant (Thermo Fisher Scientific) into the plantar surface of the right hind paw and allowed to recover. Behavior testing resumed 24 hours post-CFA injection at which point the animals received a 20 μL intraplantar injection of 100 μM (3.154 μg total) lipidated peptidomimetic immediately after the conclusion of day 1 behavioral testing. In order to minimize experimental error between groups, each group of animals received CFA from previously unopened, vacuum-sealed glass ampules ensuring CFA of identical specific activity.

Incisional post-operative pain model: To model post-operative pain, an established rat incisional model was used. In short, male and female rats were randomized into either experimental or control groups. On the day of surgery, the animals were anesthetized and placed into a prone position. Once the animal was under a surgical plane of anesthesia, a 200 μL intraplantar injection of 100 μM (31.54 μg total) lipidated peptidomimetic was made into the ipsilateral hind paw. Afterwards, the animals were returned to their home cage and allowed to recover. On the same day, 6 hours after the pre-injection, the animals were anaesthetized, placed into a prone position, and prepared for incision injury. The ipsilateral hind paw was sterilized using successive swabs of chlorohexidine, 70% ethanol, and iodine. Then, using a size 10 scalpel, a 1 cm long incision was made into the plantar surface of the ipsilateral hind paw. Short, yet firm, strokes were used to make incisions through the skin, fascia, and muscle of the hind paw. Following incision, two 50 μL injections, containing 100 μM (7.885 μg per injection) of the lipidated peptidomimetic, were made into each “half” of the incised plantar muscle. Following injection into the muscle, the skin was sutured using 6/0 silk sutures (Ethicon) in a continuous manner to discourage removal of sutures. Upon conclusion of suturing, four 25 μL injections containing 100 μM (3.9425 μg per injection) of the lipidated peptidomimetic, were made into a “quadrant” adjacent to the incision. Finally, the animals were returned to their home cage and allowed to recover for at least 16 hours.

Thermal Sensitivity Testing: Prior to testing, animals were allowed to habituate to the testing room for 1 hour on each day. Animals were placed on an enclosed elevated frosted glass platform (Ugo Baseline) and allowed 30 minutes for habituation. Once exploratory behavior ceased, an automatic Hargreaves apparatus was maneuvered (Ugo Basile) underneath the hind paw(s) of the animals. Paw withdrawal latency was calculated as the average of four trials per hind limb. Each trial was followed by a 5-minute latency period to allow adequate recovery time between trials.

Mechanical Sensitivity Testing: Each day, animals were placed on an enclosed elevated wire-mesh platform (Ugo Basile) and allowed 1 hour to habituate to their enclosure. For mice, Touch Test® Sensory Probes (Stoelting) were applied to the plantar surface of the contralateral and ipsilateral hind paw. Filaments were applied in an ascending or descending order following the Simplified Up-Down method (SUDO) for mechanical nociception testing. In short, the middle filament of the series was presented to the hind paw of the animal. If a response was elicited, the next lowest filament in the series was presented. If no response was elicited, the next highest filament in the series was presented. This method of filament presentation was repeated 5 times, with the 5^{th} filament presentation being the last one. Then an adjustment factor was added to the filament value and the force of paw withdrawal was calculated utilizing a series of conversion equations. Each paw per animal was given a 5-minute latency period between filament presentations to reduce the chance of sensitization in the paw.

Mechanical sensitivity testing on rats was conducted using an automated Dynamic Plantar Aesthesiometer (Ugo Basile). Rats were placed in an elevated enclosure atop a wire mesh platform. On each testing day, rats were given 1 hour to habituate to the room and the chamber. Testing was conducted in a manner similar to the mice, however, an automatic probe affixed with a mirror was used. The probe was set to exert a maximum upward force of 50 grams over a span of 20 seconds. The force necessary to elicit a response (as measured by swift removal of the paw from the probe) was recorded as a trial. Each animal received at least 5 minutes in between recordings to minimize sensitization. Each hind paw was tested a total of 5 times per animal.

Immunofluorescent Staining: Animal tissue was collected following a standard transcardial perfusion protocol, as previously described. Slices for staining were made at 15 microns for DRGs (mouse and human), and 50 microns for the hind paws. Mouse DRG (mDRG) tissue were affixed to charged Superfrost microscope slides (Fisherbrand). The sections were first washed 3 times with PBS, and then incubated overnight in blocking media (10% Normal Goat Serum, 3% Bovine Serum Albumin, and 0.025% Triton X-100 in PBS). The next days, the slides were incubated, overnight, in primary antibodies (Mouse anti-CGRP; 1:500 Abcam, Rabbit anti-AP2α2 1:500 Abcam). The next day, the slides were incubated with the secondary antibodies (Goat anti-rabbit 546 1:1000 Invitrogen, Donkey anti-Mouse 488 Abcam). The following day, the slides were rinsed 3 times with PBS and incubated with an IB4-647 conjugate (Invitrogen) at room temperature for 2 hours. Afterwards, the slides were rinsed twice more and mounted using ProLong™ Glass Antifade Mountant (Invitrogen).

Human L5 dorsal root ganglia (hDRGs) were purchased from Anabios. The donor was 49 years old, female, and had unremarkable past medical history. The study was certified as exempt by the University at Buffalo Internal Review Board because the hDRGs were collected from a donor and no identifying information was shared with the researchers. The hDRGs were initially preserved in formaldehyde and shipped on dry ice in 70% ethanol. Upon arrival, the hDRGs were rehydrated, sequentially, in decreasing ratios of PBS to water: 24 hours in 50% PBS then 24 hours in 30% PBS. Following rehydration, the hDRGs were cryoprotected in 30% sucrose at 4° C., and submerged in tissue freezing media (Electron Microscopy Sciences) and frozen using dry ice chilled 2-methylbutane. Once the resulting blocks were thoroughly frozen, they were placed into a −80° C. freezer for 48 hours. Cryosections were taken and mounted onto charged Superfrost microscope slides. hDRGs were sectioned and stained in a similar manner described above for the mDRGs using the same antibody concentrations.

Hind paws were stained as free-floating sections and probed in a similar manner described for DRG tissue. The following primary antibodies were used where applicable: mouse anti-HA primary antibody (1:500 Abcam) and mouse anti-CGRP (1:500 Abcam). The secondary antibody used in both instances was a goat anti-mouse 555 secondary antibody (1:1000 Abcam). After washing the secondary antibody, the sections were incubated in increasing amounts of thiodiethanol (TDE). TDE acts a tissue clearing agent aiding in fluorescent signal penetration. The first incubation consisted of 10% TDE in a 1:1 solution of PBS in ddH₂O overnight. The second incubation was in 25% TDE in 1:1 PBS in ddH₂O overnight. The third incubation was in 50% TDE in 1:1 PBS in ddH₂O overnight. The final incubation was in 97% TDE in 1:1 PBS in ddH₂O. Following the final TDE immersion, the sections were rinsed once with 1:1 PBS in ddH₂O and mounted onto charged Superfrost microscope slides using ProLong™ Glass Antifade Mountant.

All slides were allowed 24 hours to set, at 4° C., before imaging. All images were acquired using a Leica DMi 8 inverted fluorescent microscope equipped with a sCMOS Leica camera (Lieca) and connected to a HP Z4 G4 Workstation (HP) loaded with THUNDER enabled LAS X imaging software. All images were analyzed using a separate HP Z4 G4 workstation that was loaded with the LAS X imaging software. Images were exported and further modified (I.e. addition of scale bars, heat-map transformations) using ImageJ (NIH) and compiled into files using Adobe Illustrator (Adobe).

Electrophysiology: Glass electrodes were pulled using a vertical pipette puller (Narishige Group) and fire-polished for resistances of 5-8 MΩ. Current-clamp recordings were performed on dissociated adult DRG neurons from mice in vivo transfected with either scrambled control shRNA or α2 targeted shRNAs. Adult mouse neurons were dissociated as previously described. Electrophysiology experiments were conducted as previously described. Dissociated neurons were incubated with Alexa fluor-488 conjugated IB4 (Invitrogen 121411) for 5 minutes, washed thrice with sterile PBS before recordings began. Only non-fluorescing small- and medium-sized DRG neurons were recorded. Firing frequency was examined by injecting a supra threshold stimulus of 400 pA for 1000 ms. A pipette solution consisting of 124 mM potassium gluconate, 2 mM MgCl2, 13.2 mM NaCl, 1 mM EGTA, 10 mM HEPES, pH 7.2, was used. A bath solution consisting of 140 mM NaCl, 5.4 mM KCl, 1 mM CaCl₂), 1 mM MgCl2, 15.6 mM HEPES, and 10 mM glucose, pH 7.4, was used. All data were acquired using Multiclamp-700B (Molecular Devices), digitized, and filtered at 2 kHz. Data acquisition was monitored and controlled using pClamp 10.2 and analyzed using Clampex (Molecular Devices).

Western Blot Analysis: Total protein was collected from DRG tissue collected from animals following experimentation. DRGs were homogenized in chilled RIPA buffer containing a protease inhibitor (Sigma) and stored at −80° C. until needed. All samples were run on Mini-PROTEAN TGX Precast Gel (Bio-Rad) and transferred to a 0.45 m nitrocellulose membrane (BioRad). Membranes were probed overnight at 4° C. with rabbit anti-AP2α2 (1:1000, Abcam) or rabbit anti-3-Actin (1:1000, Sigma) in 5% bovine serum albumin (BSA) prepared in 1× tris-buffered saline-tween (TBST). On the following day, membranes were washed three times for five minutes in 1×TBST before being incubated for 1 hour at room temperature in a secondary anti-rabbit horseradish peroxidase conjugate antibody (1:5000; Promega) prepared in a 5% BSA in 1×TBST solution. After secondary anti-body incubation, the membrane was washed more three times for five minutes per wash before being developed and imaged. Bands were visualized with enhanced chemiluminescence on a Chemidoc Touch Imaging System (Bio-rad) and quantified with Image J Software (NIH). Each experiment was repeated at least three times.

Statistics: All statistical tests were performed using Prism (GraphPad). The data are shown as means s.e.m. Power analysis was conducted for animal experiments to achieve detection limits with an α-value set at 0.05. Statistical significance was determined utilizing a p-value<0.05 for all experiments. Repeated measures two-way ANOVA statistical tests with multiple comparisons and stringent Bonferroni correction, one-way ANOVA with Holms-Sidak correction, and student's t-test were used where appropriate. Tau analysis was conducted using the following equation: W(t)=(W₀−p)e^{-kt+p}, where ‘Wₜ’ is the withdrawal threshold at given time ‘t’, ‘W₀’ is the withdrawal threshold at t=0, ‘p’ is the plateau value, ‘k’ is rate constant, and ‘t’ is time in days. Constraints were implemented to prevent near infinite tau values; W₀>1 and p<16. For a two-phase decay fitting, the following equation was used: W(t)=p+Fe^{-at}+Se^{-lt} (Wₜ: withdrawal threshold at given time ‘t’, F: fast component of decay [F=(W₀−p)Fₚ], S: slow component of decay [S=(W₀−p)(1−Fₚ)], Fₚ: fraction of withdrawal threshold due to the fast phase, W₀: withdrawal threshold at t=0, p: plateau value, a: fast rate constant, 1: slow rate constant, t: time in days).

### Results

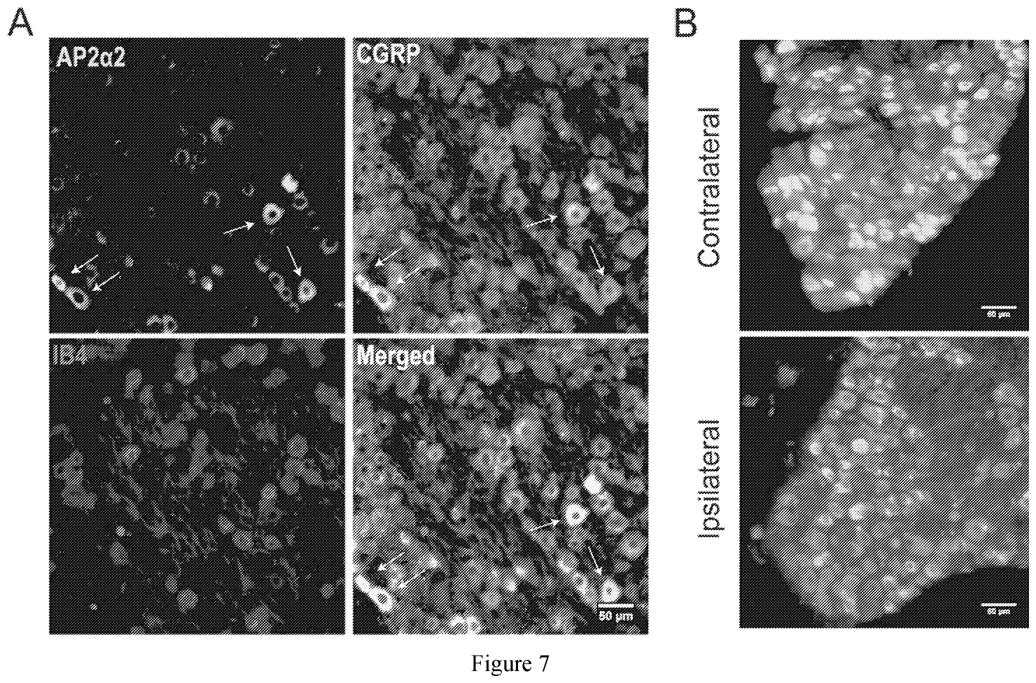

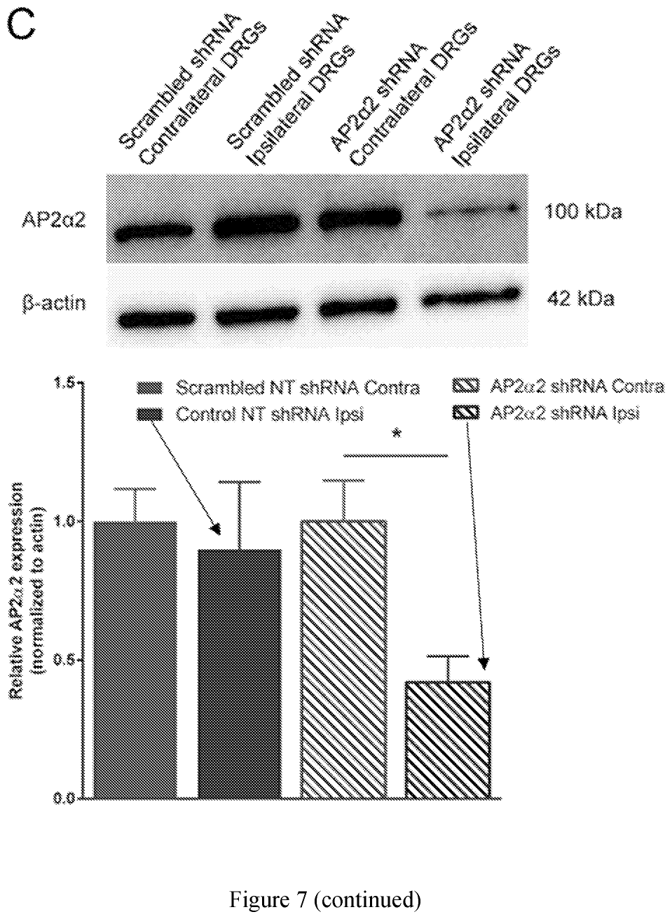

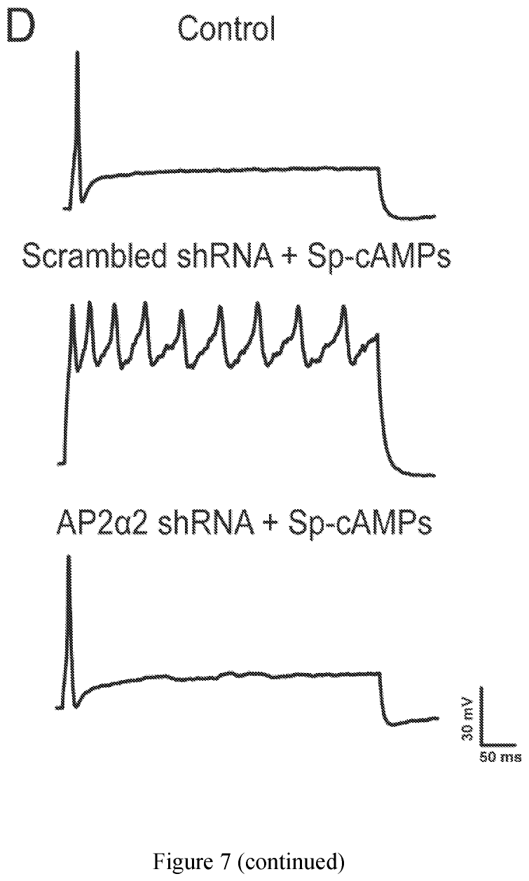

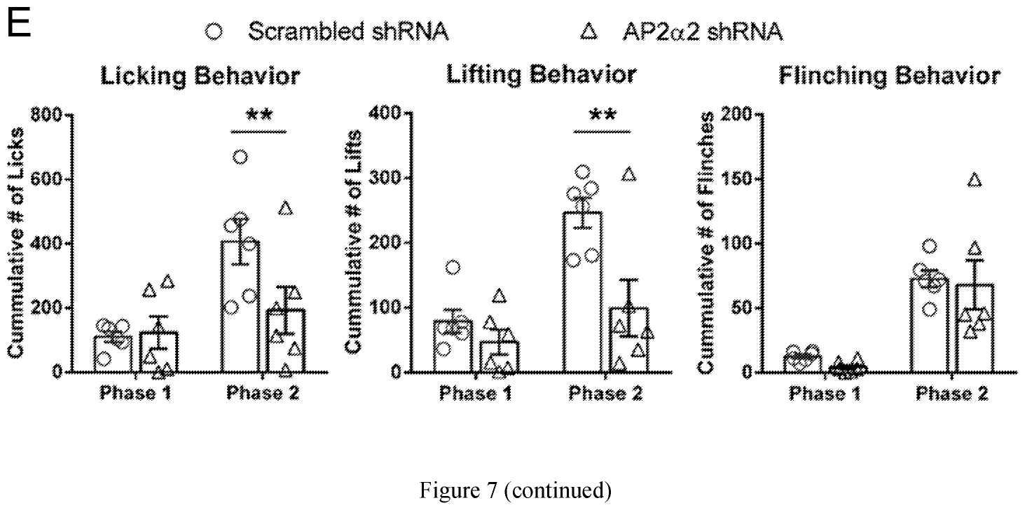

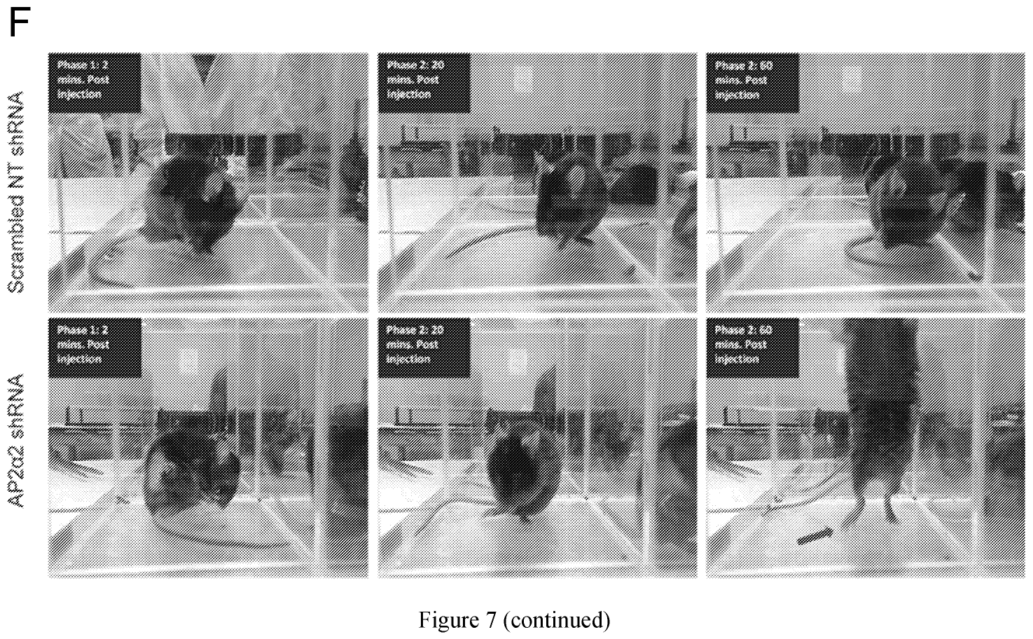

AP2α2 is preferentially expressed in CGRP containing DRG neurons: Previous immunological labeling of AP2α2 in the superficial lamina of the rodent dorsal horn suggested a putative differential expression of AP2α2 in nociceptors. In order to resolve this, mDRG neurons were probed with antibodies against AP2α2, CGRP, and an Alexa fluor-conjugated IB4. Interestingly, strong immunofluorescent co-localization between CGRP and AP2α2 was observed, while virtually no IB4⁺ neurons expressed AP2α2 (FIG. 7A). The overlapping immunoreactivity of AP2α2 and CGRP, alongside a lack of immunoreactivity in IB4⁺ neurons, suggested AP2α2 participates in peptidergic DRG neuronal signaling implicating it in thermal sensitivity during pain.

In vivo DRG neuronal AP2α2 knockdown modulates peripheral nociceptor excitability and reduces acute inflammatory pain behaviors: CGRP expression is a strong marker for thermal nociceptors due to robust co-expression of the transient receptor potential vanilloid 1 (TRPV1) ion channel. TRPV1 is known to principally govern nociceptor responses to noxious thermal and chemical sensation as well as acidic pH. Inflammation-induced ongoing pain is therefore, driven by TRPV1 nociceptive fibers. Observing a high degree of co-expression of AP2α2 and CGRP suggested that AP2α2 contributes to thermal and chemical responsiveness. To test this, a unilateral injection of shRNAs against AP2α2 were made into the sciatic nerve of C57BL/6 mice. This produced a significant decrease in AP2α2 protein expression levels 7 days post-shRNA injection (FIGS. 7B and 7C) and was sufficient in reducing PKA-induced hyperexcitability in dissociated adult DRG neurons from these mice (FIG. 7D). Dissociated contralateral IB4⁻ DRG neurons demonstrated firing accommodation under control conditions (n=10; only 2 of 10 exhibited more than 2 action potentials, FIG. 7D top). Ipsilateral IB4-DRG neurons cultured from scrambled shRNA animals displayed typical loss of firing accommodation under PKA stimulatory conditions (n=7; 5/7 hyperexcitable, FIG. 7D middle). Ipsilateral DRG neurons cultured from AP2α2 shRNA animals displayed firing accommodation (n=9; 2/9 hyperexcitable FIG. 7D bottom).

The behavioral consequence of in vivo DRG neuronal AP2α2 knockdown was first assessed using the formalin acute inflammatory pain assay. The biphasic nature of this assay offers compartmentalization of observed behavioral effects to distinct neurophysiological changes. DRG neuronal knockdown of AP2α2 did not alter transient phase 1 pain-like behaviors (FIG. 7E), however, there was a significant decrease in inflammatory phase 2—paw licking (scrambled shRNA 406±70; AP2 shRNA 193±73) and lifting behaviors (FIG. 7E; scrambled shRNA 246±23; AP2 shRNA 99±43). Additionally, there were time-dependent changes in animal resting behavior (FIG. 7F). At the start of observation, in phase 1, both groups exhibit increased paw licking behaviors, indicative of pain (FIG. 7F left). However, at the start of phase 2, the scrambled shRNA group continued to engage in paw licking, whereas the AP2α2 shRNA group engaged in grooming behavior (FIG. 7F middle). Finally, at the conclusion of observation, the scrambled group maintained paw licking behavior, while the AP2α2 group began to exhibit exploratory behavior (FIG. 7F right).

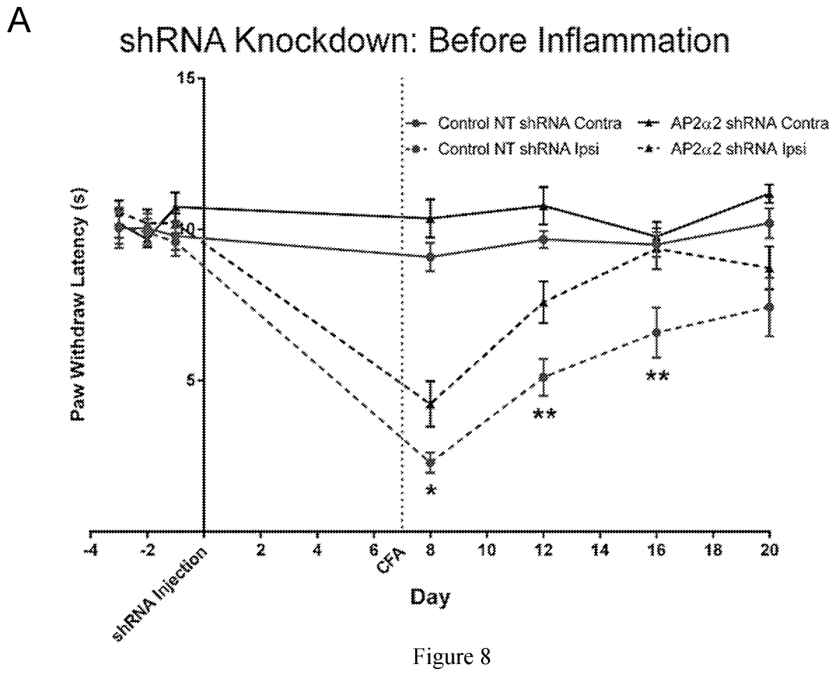

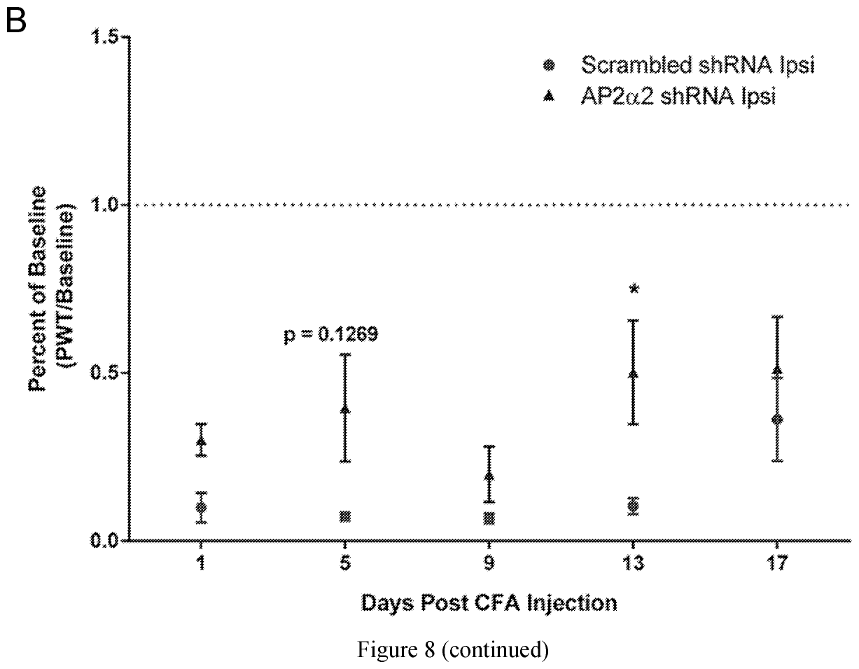

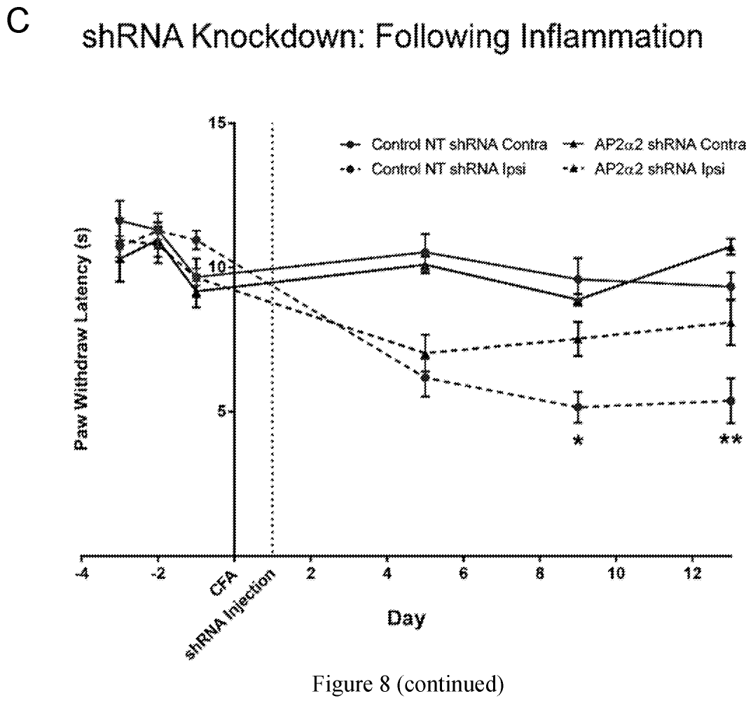

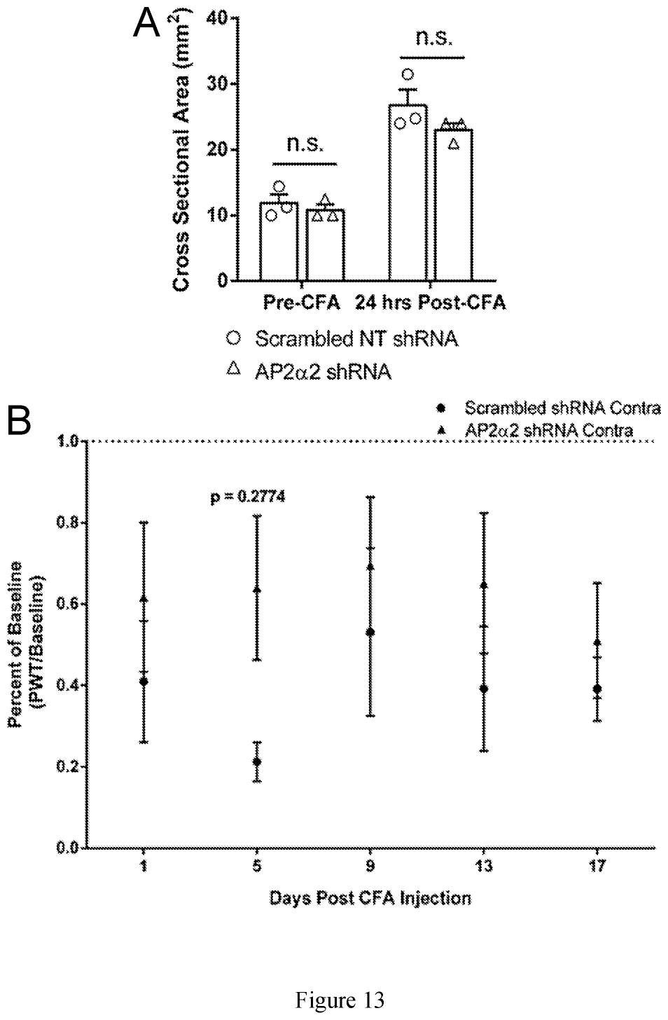

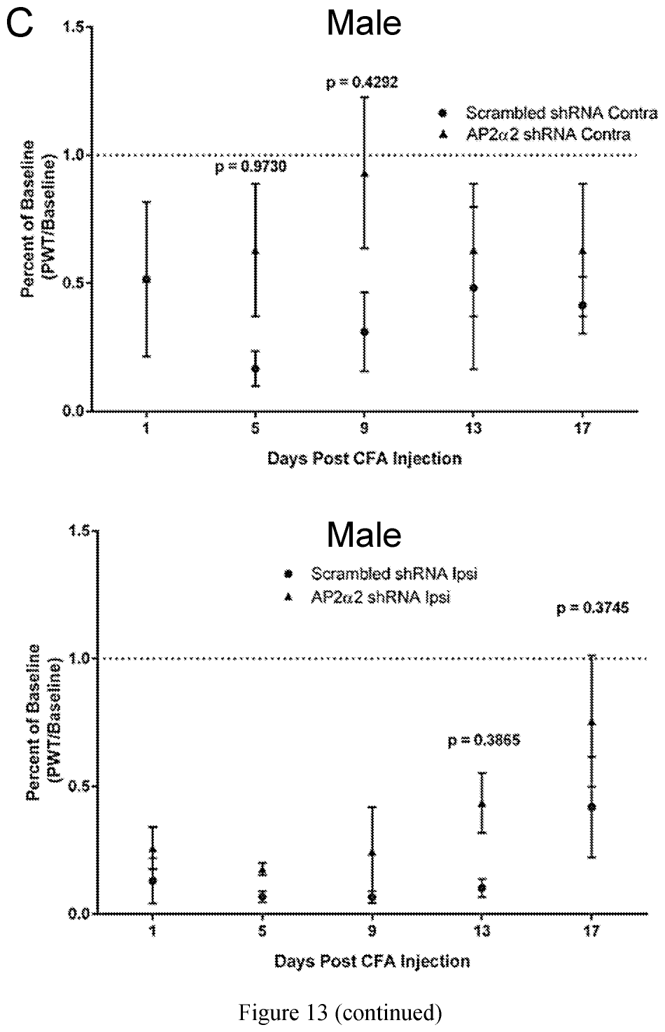

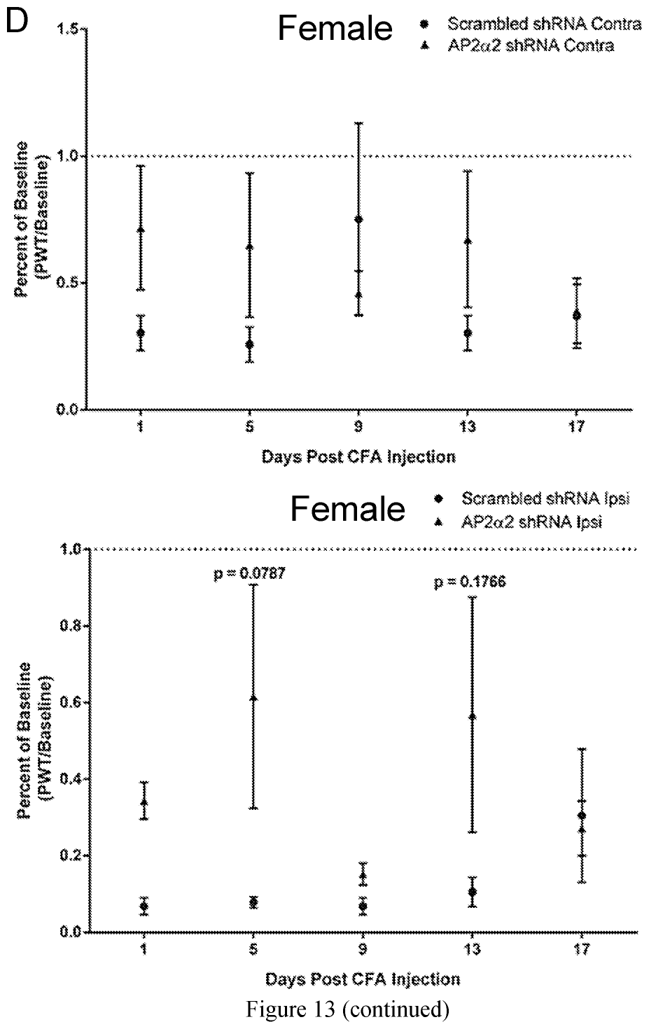

To evaluate the contribution of AP2α2 to chronic inflammatory pain, an intraplantar injection of Complete Freund's Adjuvant (CFA) was conducted. CFA induces pain and local inflammation through immune cell recruitment and activation. Using this model, the contribution of endocytosis in the development (AP2 knockdown pre-inflammation FIG. 8A) and maintenance (AP2 knockdown post-inflammation FIG. 8C) of chronic inflammatory pain signaling was evaluated. In the pre-inflammation condition, control animals (n=11) exhibited sensitivity to thermal stimuli 24 hours following CFA injection (2.3±0.3 s), whereas the AP2α2 group (n=12) showed reduced thermal sensitivity following CFA injection (4.2±0.8 s). The difference in paw withdrawal latencies persisted for the duration of experimentation until both groups displayed full recovery of thermal sensitivity. shRNA-mediated AP2α2 knockdown experiments were conducted following inflammation (FIG. 8C). AP2α2 knockdown animals recovered more rapidly (n=8, day-5: 7.0±0.6 s; day-9: 7.5±0.6 s; day-13: 8.1±0.8 s) compared to control shRNA animals (n=8, day-5: 6.2±0.7 s; day-9: 5.2±0.5 s; day-13: 5.4±0.8 s). Due to the localization of AP2α2 in peptidergic neurons, knockdown was not expected to alter mechanical sensitivity but, surprisingly, a slight reduction in mechanical sensitivity was observed when AP2α2 was preemptively knocked down, significantly at day 13 (FIG. 8B). Contralateral mechanical responsiveness data is shown in FIG. 13. These data suggested that DRG neuronal AP2α2 knockdown disrupted neuroplastic processes necessary for both thermal and mechanical sensitivity during inflammation.

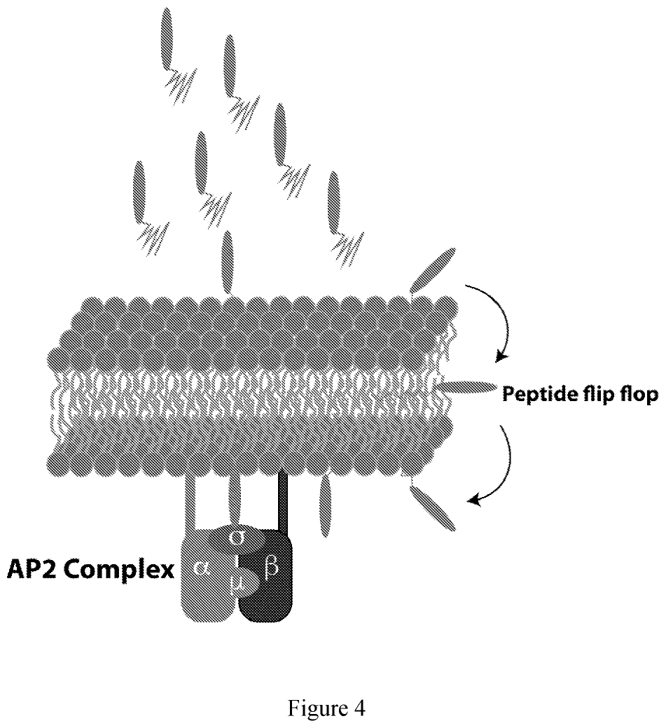

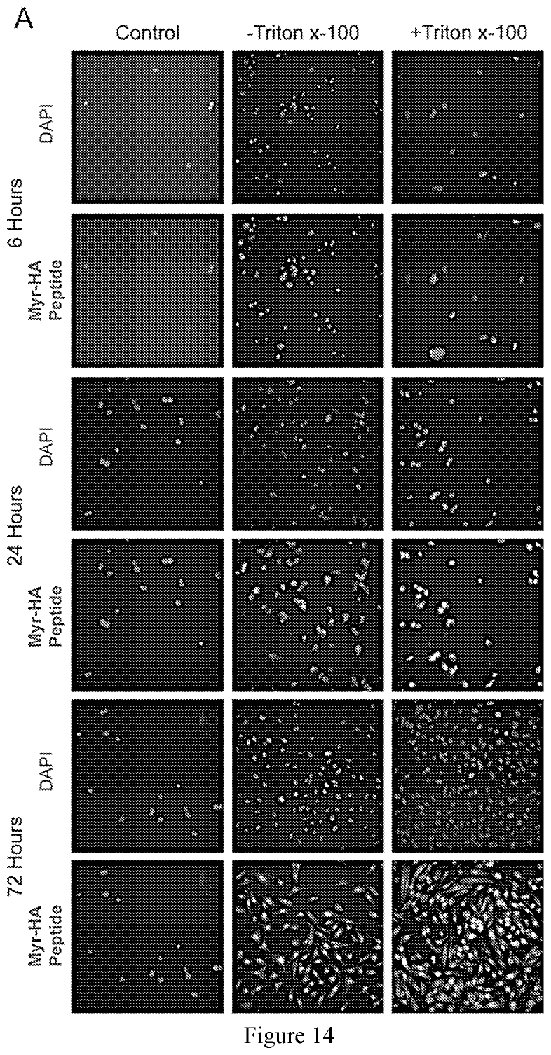

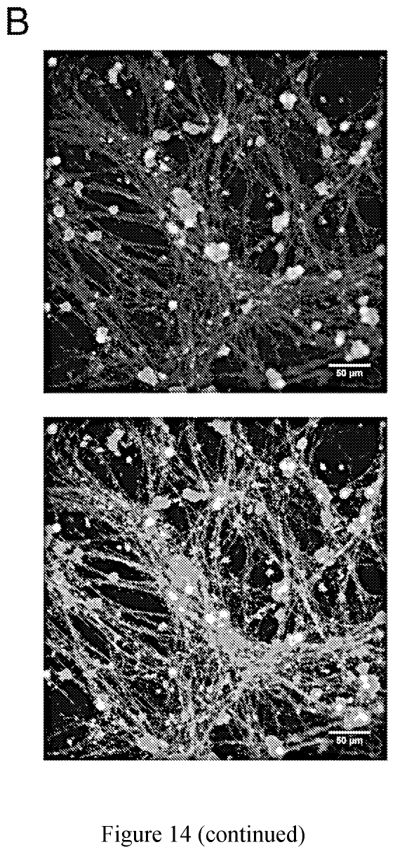

Lipidated peptidomimetics localize to lipid compartments in the rodent hind paw: Small myristoylated peptides have been previously used to target nociceptor endings and modify pain behavior. Small lipidated peptides are able to traverse the membrane by a flip-flop mechanism gaining access to the inside of the cell (FIG. 4). How lipidated peptides might enter nociceptive nerve endings and persist in cells and tissues after administration was explored. The use of a lipidated AP2 inhibitor peptide is also described. A lipidated version of the influenza hemagglutinin (HA) protein (HA-peptide) was generated and its localization was visualized by immunocytochemistry. It was found that the HA-peptide embedded into the membranes of CHO cells resulting in robust membrane labeling (FIG. 14). This was surprising considering the conditions of the experiment; exposure to HA-peptide for 3 hours followed by a series of washes and media replacement. The persistence of HA-immunoreactivity over time (at least 72 hours) was equally surprising, which suggested that small lipidated peptides maintain a degree of stability during large cellular events such as mitosis. The persistence of lipidated peptides was similarly observed in cultured DRG neurons, detected 72 hours after initial application and a series of media changes (FIG. 14).

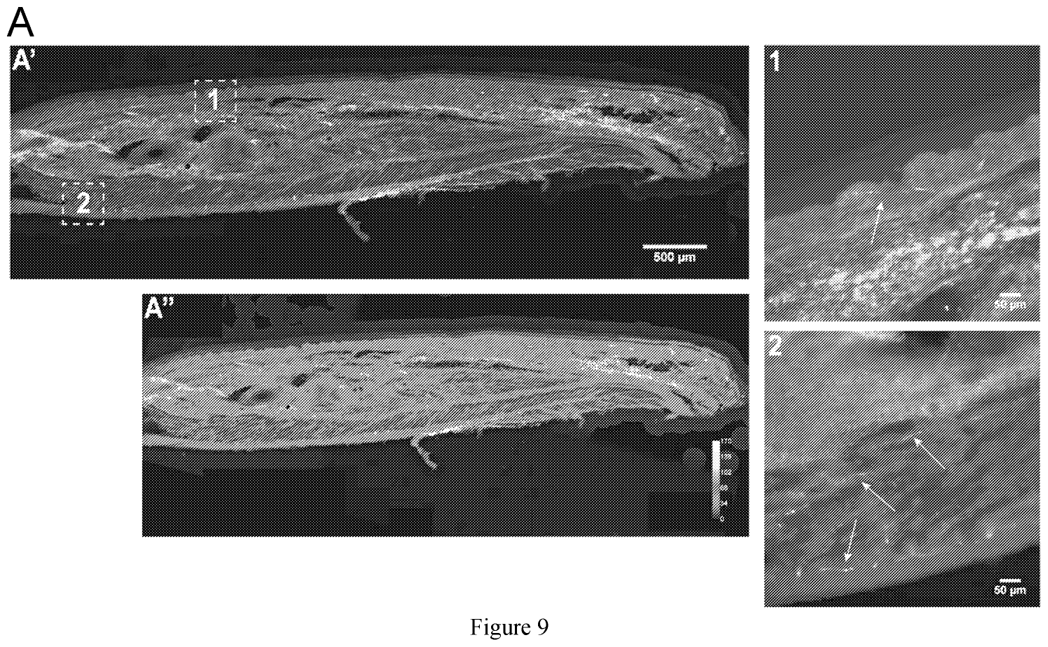

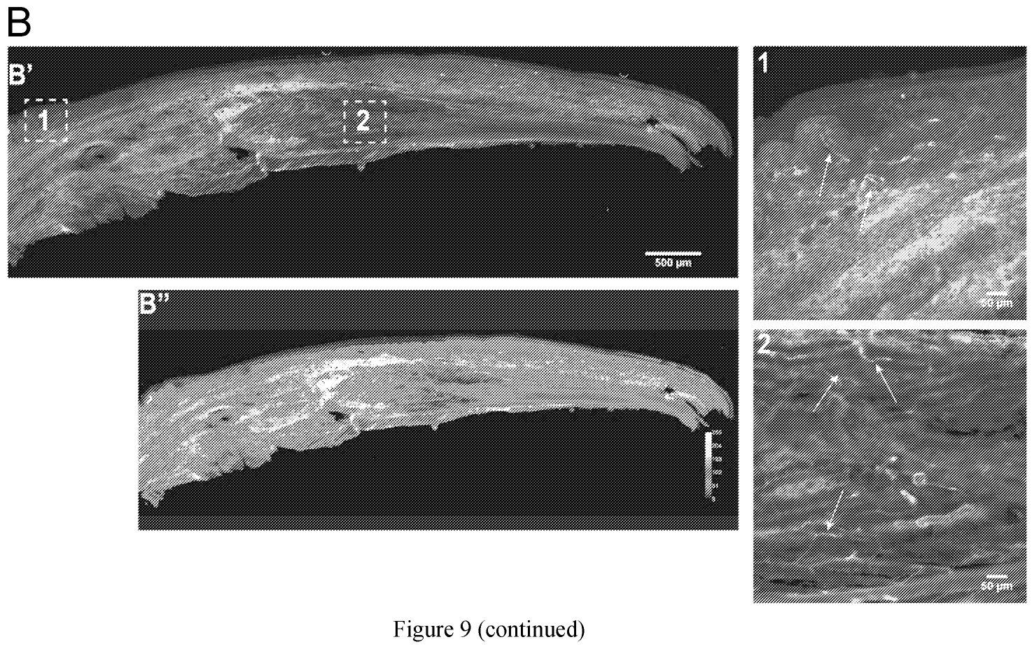

Next, it was determined whether the lipidated HA-peptide could similarly demonstrate stability when applied in vivo, and whether inflammation impacts absorption and distribution of the peptide. Injection of the HA-peptide into the hind paw of mice produced robust HA immunoreactivity within the dermis and lipid dense compartments, while the epidermis and muscle displayed weak immunoreactivity 24 hours after local injection (FIG. 9A). The presence of HA-immunoreactivity in nerve-like fibers in the dermis (FIG. 9A-1) is noted, as well as the muscle tissue (FIG. 9A-2). The presence of the HA-peptide in muscle localized nerve-like fibers suggested that lipidated peptides are able to laterally diffuse along the length of the fiber. A similar pattern of distribution was also observed under inflammatory conditions (FIG. 9B). There was considerable labeling of nerve-like fibers innervating the dermis (FIG. 9B-1) and muscle (FIG. 9B-2) under non-inflammatory conditions. Under inflammatory conditions we noted more intense global immunoreactivity (FIG. 9B″).

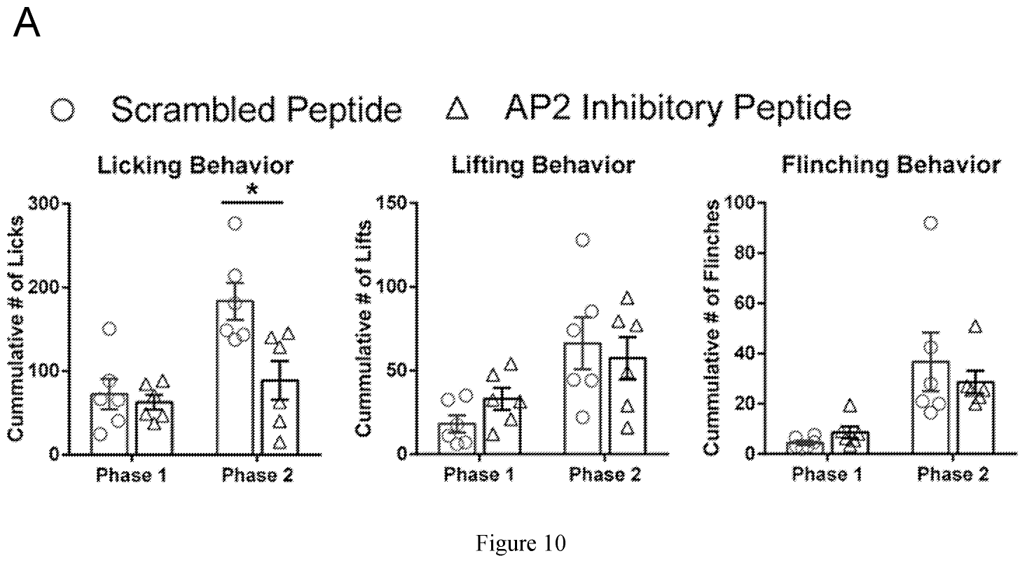

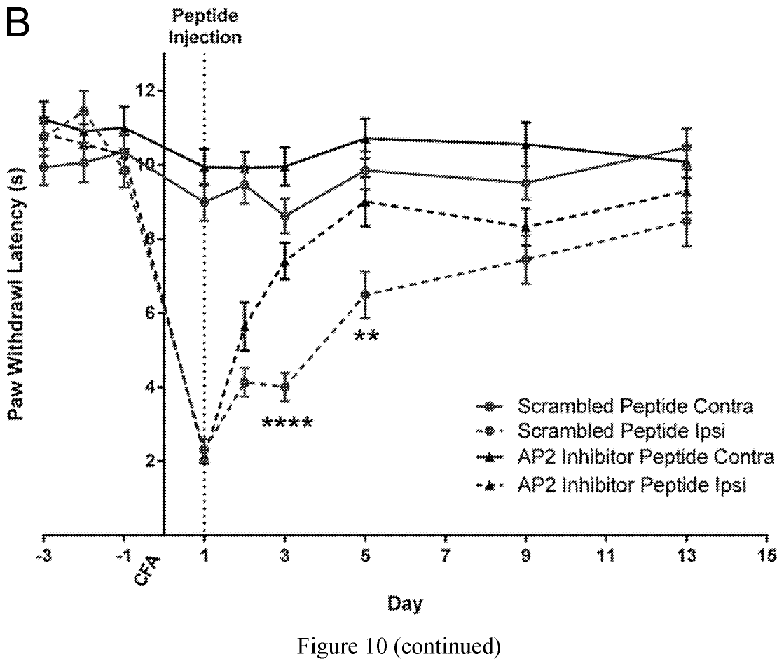

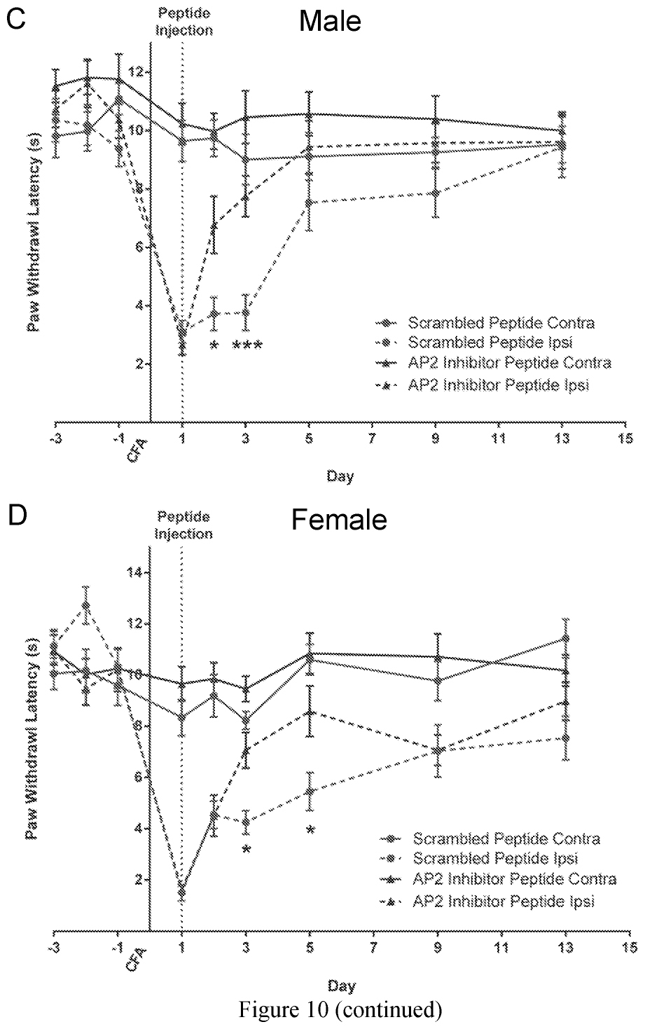

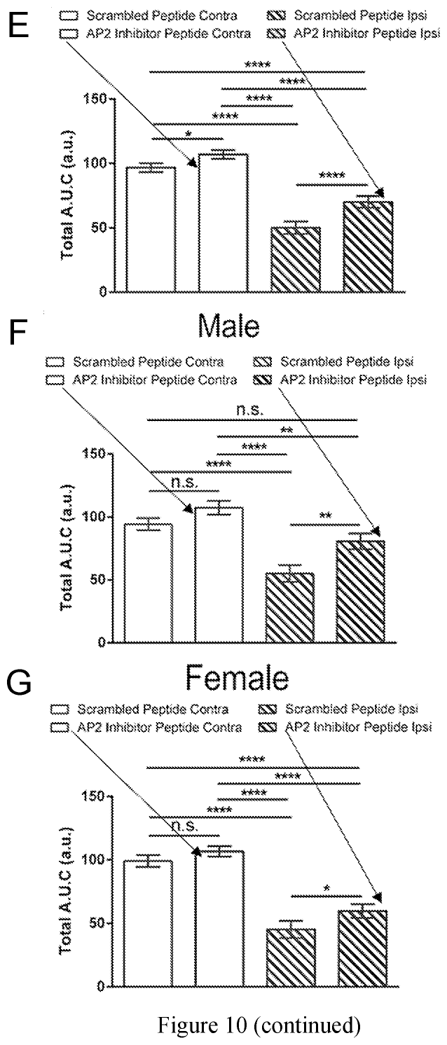

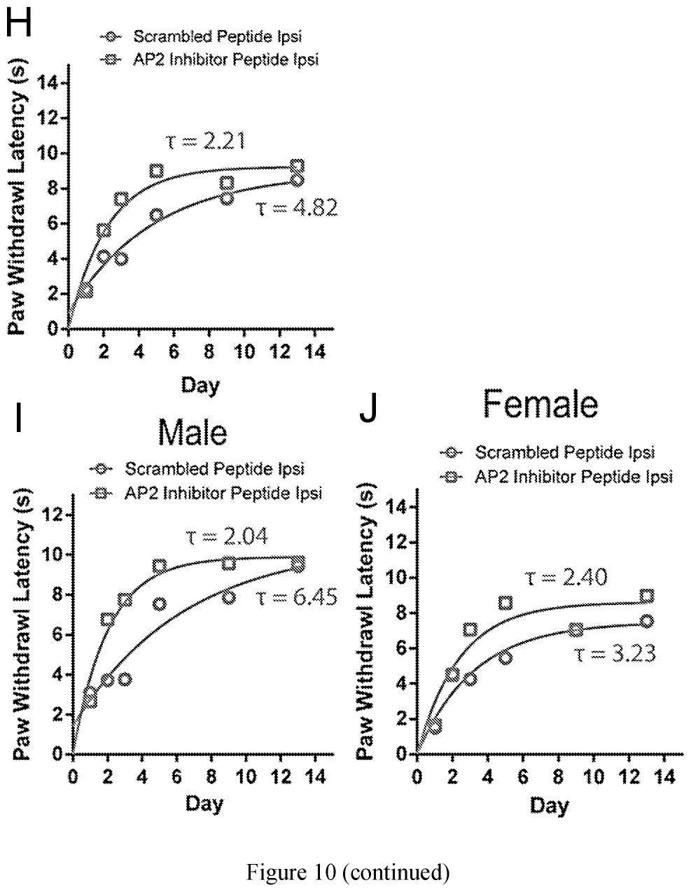

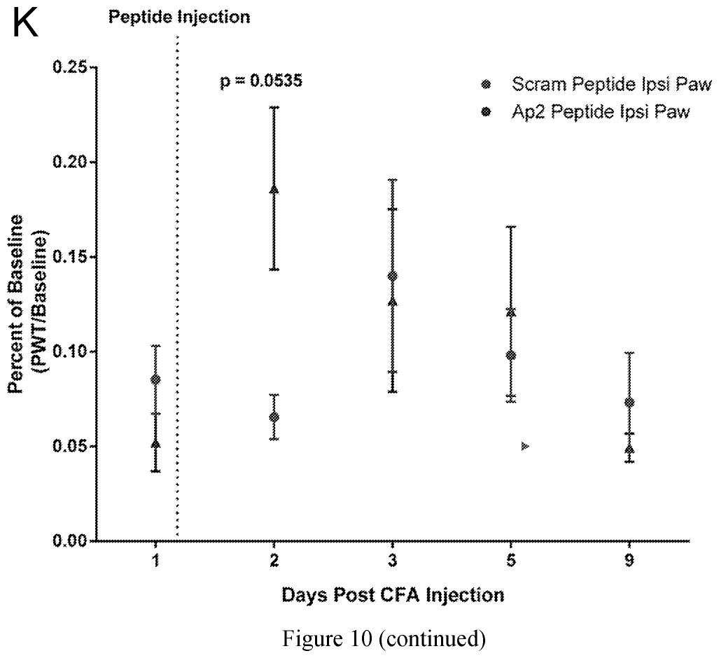

AP2 Inhibitory peptide attenuated pain behaviors during inflammation: The consequences of pharmacologically inhibiting endocytosis in peripheral nociceptor afferents during inflammation was assessed using a small lipidated peptide AP2-CME inhibitor. A short peptide derived from the human CD4 di-leucine motif with a myristoyl moiety conjugated to the N-terminal (Table 1) was unilaterally injected 24 hours before administering the formalin assay. This peptide sequence was shown to have high affinity (650 nM) for the AP2 complex. One-time injection of the lipidated AP2 inhibitory peptide produced a robust decrease in cumulative phase 2 paw licking behavior (scrambled peptide n=6, 184±22; AP2 inhibitory peptide n=6, 89±23) while other measures of pain-like behavior remained relatively unchanged (FIG. 10A). Representative videos of this behavior are provided for reference. From this, some of the reduced pain behaviors observed AP2α2 knockdown experiments were recapitulated, reinforcing the premise that local nociceptor endocytosis is participating in the development of inflammatory pain.

The analgesic potential of the AP2 inhibitory peptide during established CFA-induced inflammatory pain was studied. First, CFA inflammation for 24 hours was induced and then a simple one dose injection of peptide was delivered directly into the inflamed paw. This single injection of the AP2 inhibitory peptide produced a persistent increase in paw withdrawal latency that lasted for 4 days (n=8, day-1: 2.2±0.3 s; day-2: 6.1±0.8 s; day-3: 7.3±0.6 s; day-5: 8.3±0.7 s; day-9: 8.0±0.6 s), whereas the scrambled peptide group (n=8, day-1: 2.4±0.3 s; day-2: 3.8±0.5 s; day-3: 3.8±0.4 s; day-5: 5.5±0.6 s; day-9: 7.0±0.8 s) displayed the stereotypical thermal responsiveness recovery curve of this assay (FIG. 10B). Interestingly, after segregating the data by gender, an unexpected sex-dependent temporal component to the onset of analgesia was noted (FIGS. 10C and 10D). In male mice (scrambled peptide; n=4, AP2 inhibitory peptide; n=4), there was a more immediate response to the peptide (FIG. 4C), whereas female mice (scrambled peptide; n=4, AP2 inhibitory peptide; n=4), exhibited a delayed onset of action (FIG. 10D). Area under the curve (A.U.C.) quantification revealed that animals grouped together experienced analgesia to the AP2 inhibitor peptide (FIG. 10E) and segregating the data revealed that the AP2 inhibitory peptide was able to produce an analgesic-like effect in both males (FIG. 10F) and females (FIG. 10G). To further understand the gender differences in thermal recovery, a method was devised to express the rate of recovery as a time constant. Since thermal sensitivity exhibits time-dependent recovery in CFA models, the scrambled peptide group recovery phase (day 1-day 11) was selected as a measure of unassisted resolution of thermal sensitivity. By fitting this curve to a first order exponential decay equation, a time constant, tau (τ), was calculated. Although unconventional, quantification of a tau allowed for a more comprehensive understanding of the kinetics of thermal recovery following single-dose administration, important for the development of clinically-relevant novel analgesics. Application of the AP2 inhibitor peptide resulted in a more rapid thermal sensitivity recovery (τ_{AP2=2.21}) compared to control (τ_{control}=4.82; FIG. 10H). Segregation of the original data by sex uncovered a difference between male and female recovery during inflammation in both scrambled and AP2 inhibitor peptide groups; male mice experienced a robust decrease in tau (FIG. 41; τ_{control}=6.45, τ_{AP2}=2.04), while female mice exhibited a modest decrease in tau (FIG. 4J; τ_{control}=3.23, τ_{AP2}=2.40). Administration of the AP2 inhibitory peptide only slightly affected mechanical sensitivity almost reaching significance 24 hours following application. All other time points were indistinguishable suggesting that pharmacological inhibition of endocytosis primarily targets thermal sensitivity (FIG. 10L) but might have consequential indirect effects on mechanical sensitivity.

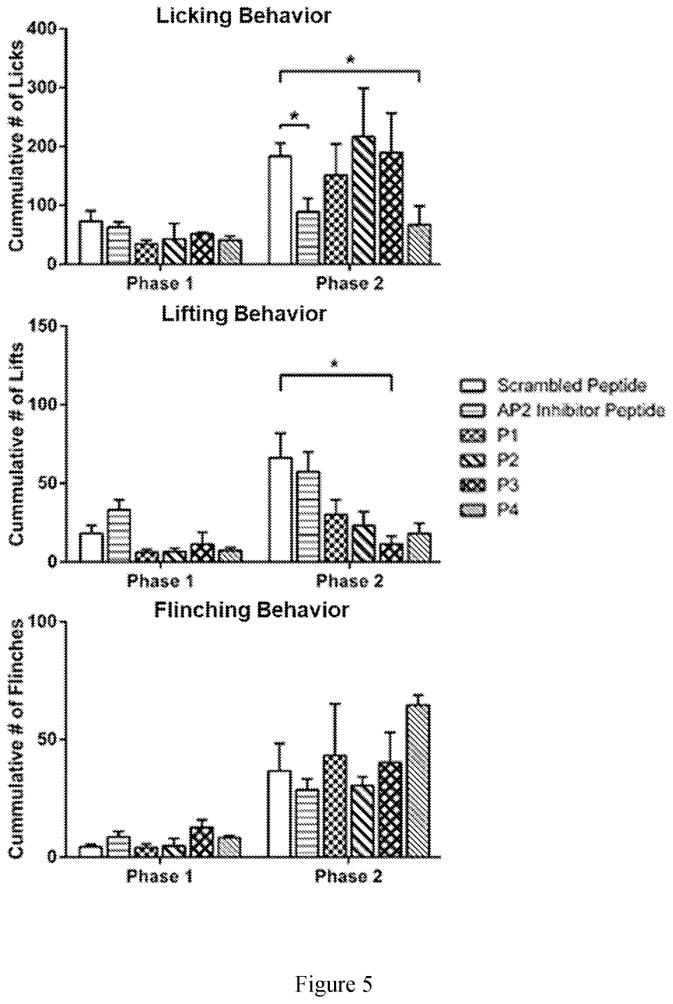

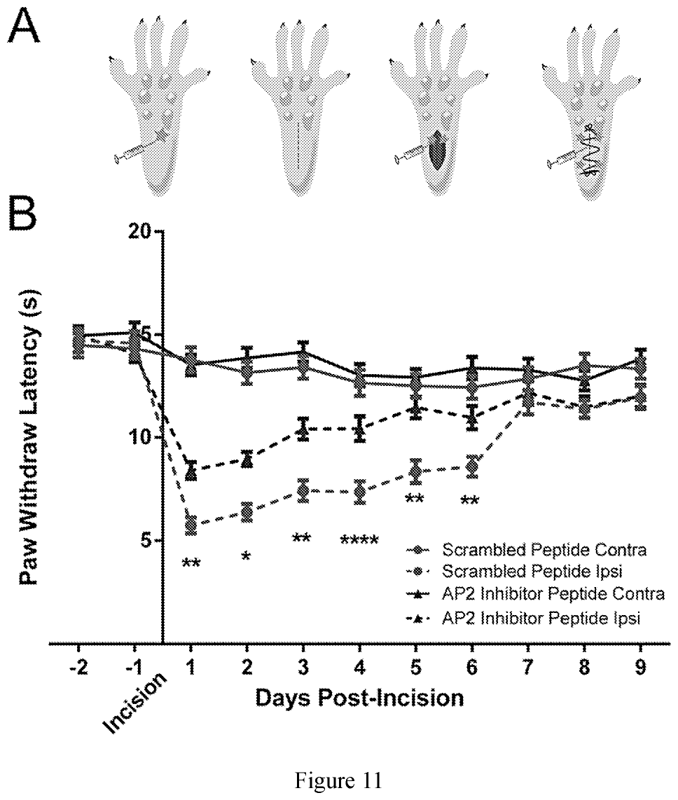

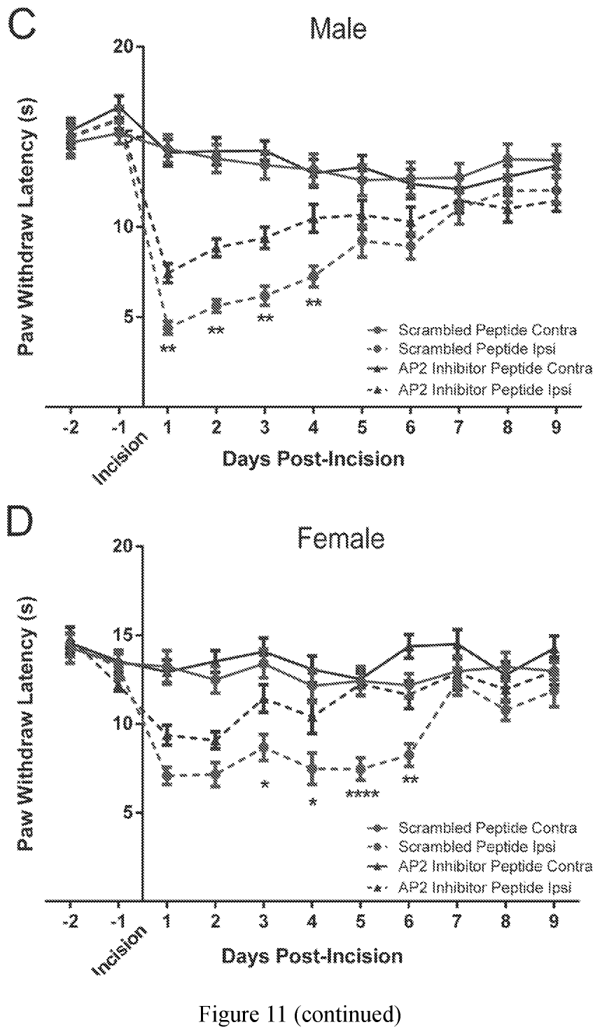

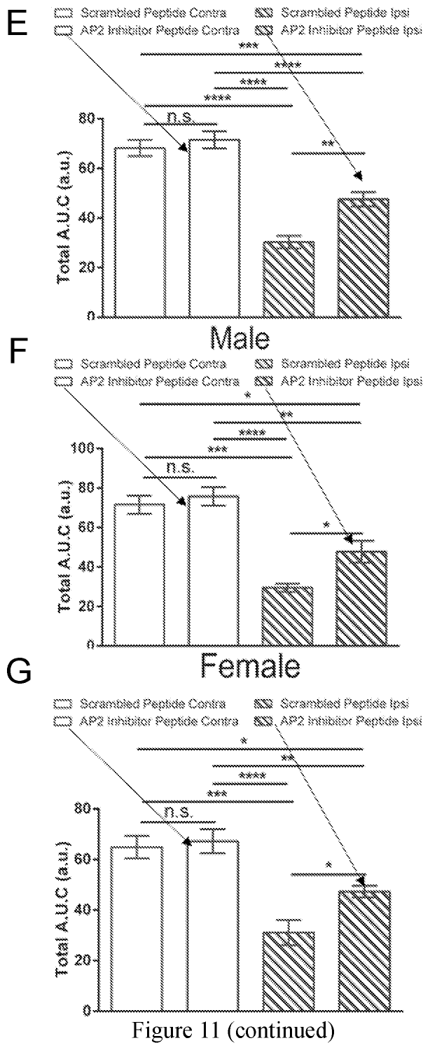

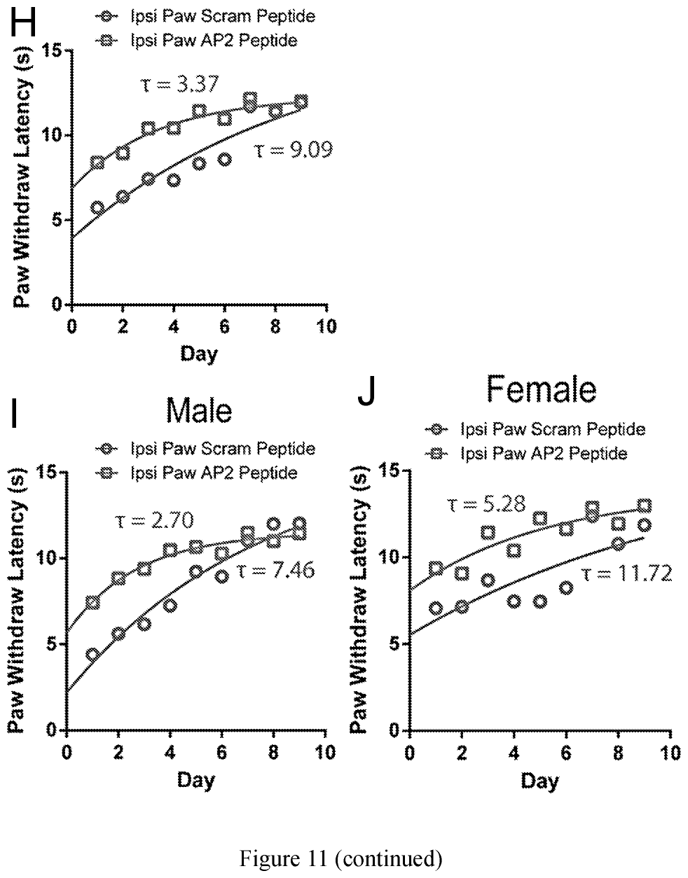

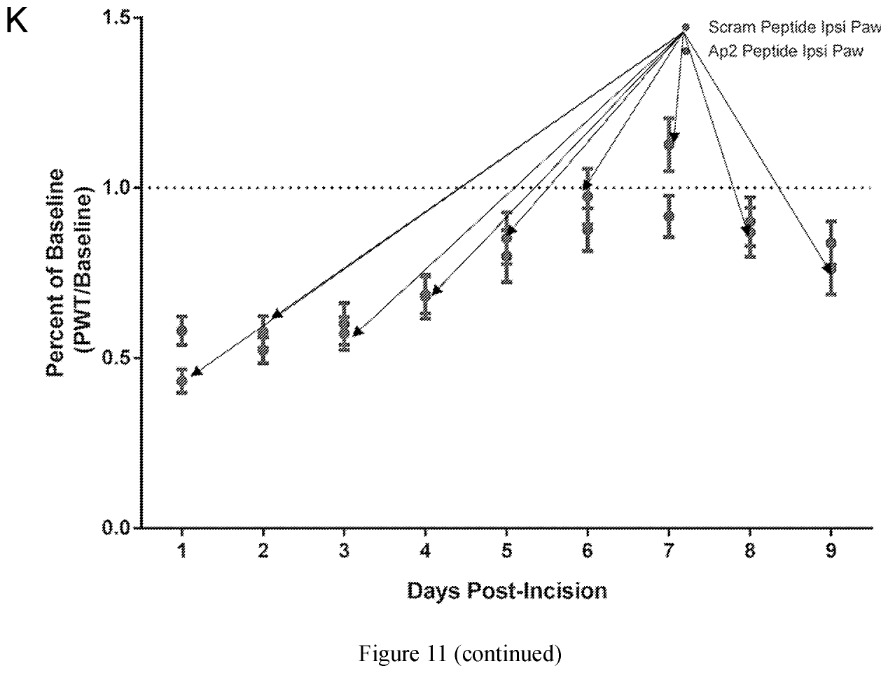

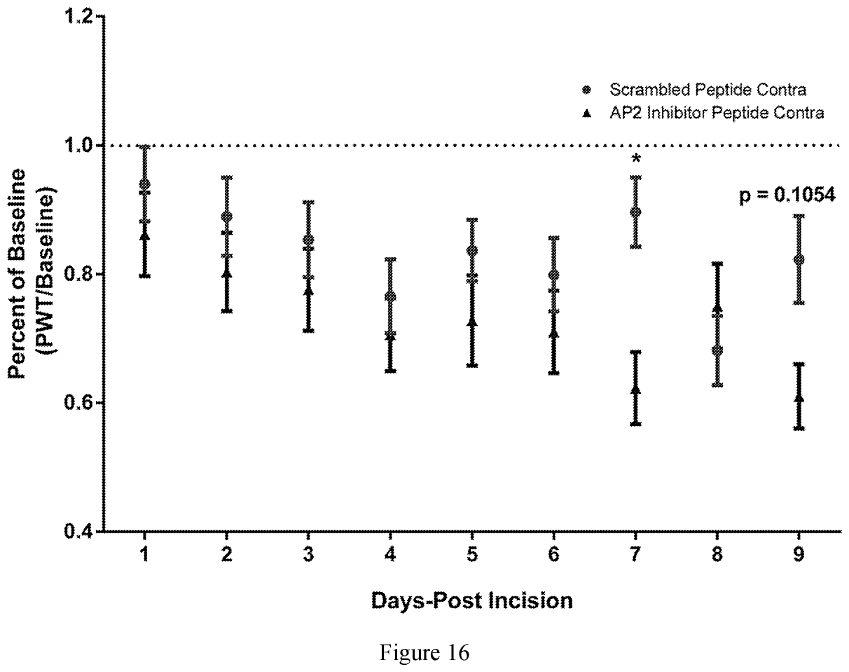

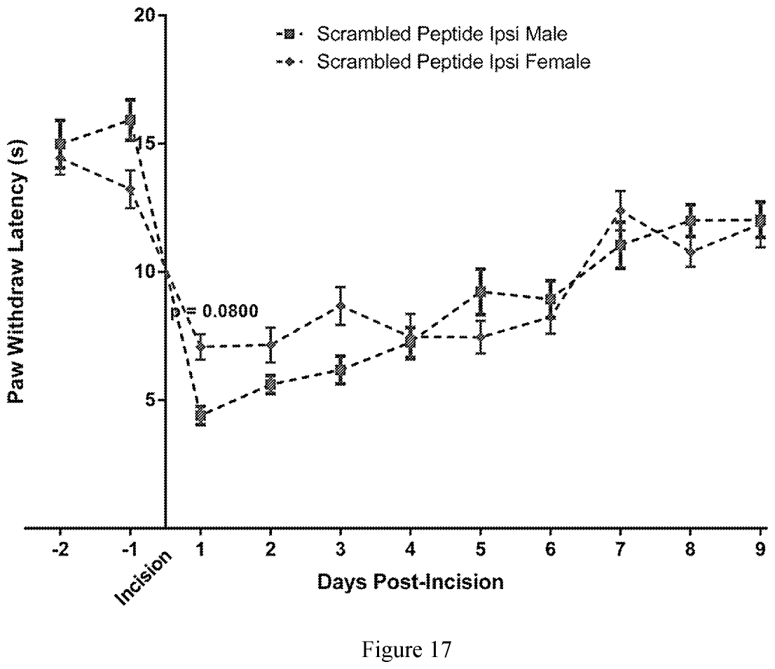

In addition to chemogenic-induced inflammation, also explored was the analgesic potential of the AP2 inhibitory peptide in an injury-induced inflammation/rat post-operative pain model. Preclinical incision models are useful for determining the efficacy of pharmacologic treatment during the early postsurgical phase. For this assay, a potential clinical application schedule was simulated for the AP2 inhibitory peptide; sub-cutaneous administration into the hind paws of rats 6 hours before incision, and then a series of smaller sub-cutaneous and intra-muscular injections immediately following incision (FIG. 11A). Application of the AP2 inhibitory peptide (n=12) produced a profound and long-lasting reduction in thermal sensitivity compared to the scrambled peptide (n=8; FIG. 11B). Just as in previous models, the AP2 inhibitory peptide was capable of increasing thermal sensitivity thresholds for the duration of the experiment following a single application (scrambled peptide day-1: 5.7±0.4 s; day-2: 6.4±0.4; day-3: 7.4±0.5 s; day-4: 7.4±0.5 s; day-5: 8.3±0.6 s; day-6: 8.6±0.5 s; day-7: 11.7±0.6 s; day-8: 11.4±0.4 s; day-9: 12.0±0.6 s vs AP2 inhibitory peptide day-1: 8.4±0.4 s; day-2: 9.0±0.3; day-3: 10.4±0.5 s; day-4: 10.4±0.6 s; day-5: 11.4±0.5 s; day-6: 11.0±0.6 s; day-7: 12.1±0.6 s; day-8: 11.5±0.5 s; day-9: 12.0±0.5 s). As noted in the CFA model, there was an apparent sex-dependent response to the AP2 inhibitory peptide after the data was segregated by gender. First, in the scrambled group, males had faster withdrawal latencies than females 24 hours after injury (FIG. 16). Second, male rats displayed a progressive recovery after incision injury as similarly reported for male mice, while females demonstrated a thermal responsiveness that remained relatively constant over 6 days followed by a rapid return to baseline at day 7 (FIG. 11D; FIG. 17). In male rats injected with the AP2 inhibitory peptide, a significant reduction in thermal sensitivity but a relatively similar recovery pattern was observed (FIG. 11C). Remarkably, in females, the AP2 inhibitor peptide caused thermal responsiveness to return to baseline by as early as day 3 (FIG. 11D). A.U.C. quantification revealed that the AP2 inhibitory peptide was capable of increasing the A.U.C. indicative of an analgesic-like effect compared to the scrambled peptide (FIG. 11E). This effect was conserved when separating the data based on sex; the AP2 inhibitory peptide produced an analgesic-like effect in both males (FIG. 11F) and females (FIG. 11G). Additionally, the AP2 inhibitor peptide was capable of increasing the rate of recovery following incision (FIG. 11H; τ_{control}=9.09, τ_{AP2}=3.37). In this parameter, both sexes showed increased recovery (Male: FIG. 5I; τ_{control}=7.46, τ_{AP2}=2.70, Female: FIG. 11J; τ_{control}=11.72, τ_{AP2}=5.28). Although there was a significant effect on thermal sensitivity, there was no significant change in ipsilateral mechanical sensitivity (FIG. 11L).

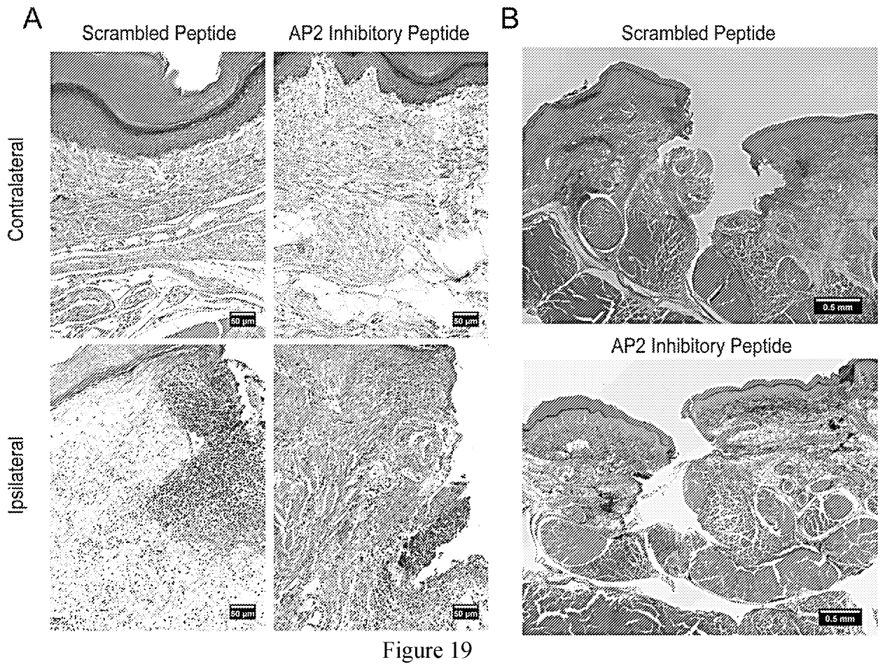

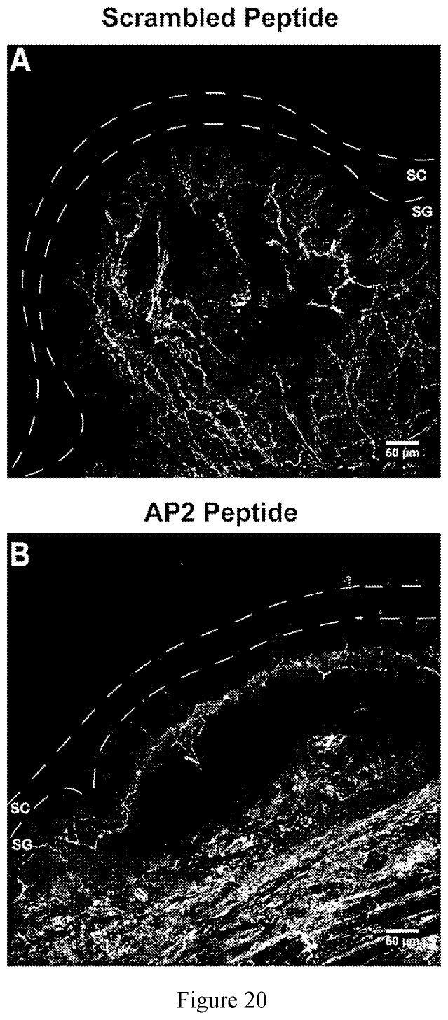

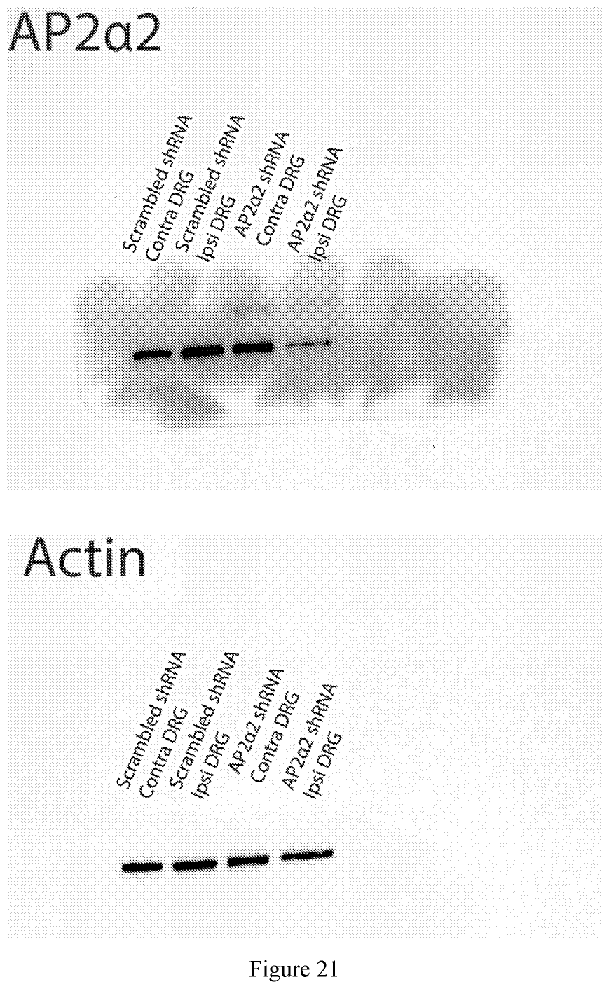

The efficacies di-leucine based peptides derived from other human proteins were tested and sequence-dependent reductions in various nocifensive behaviors were observed (FIG. 18). However, afferents at the site of formalin injection receiving the highest concentrations of formalin, hence are more likely to undergo fixation, inactivation and/or desensitization. Therefore, the formalin assay inherently underestimates the analgesic potential of lipidated peptides designed to penetrate afferent endings. Genetic and pharmacological inhibition of endocytosis did not preclude edema (FIGS. 13 and 15) nor immune cell activation and infiltration (FIG. 19).

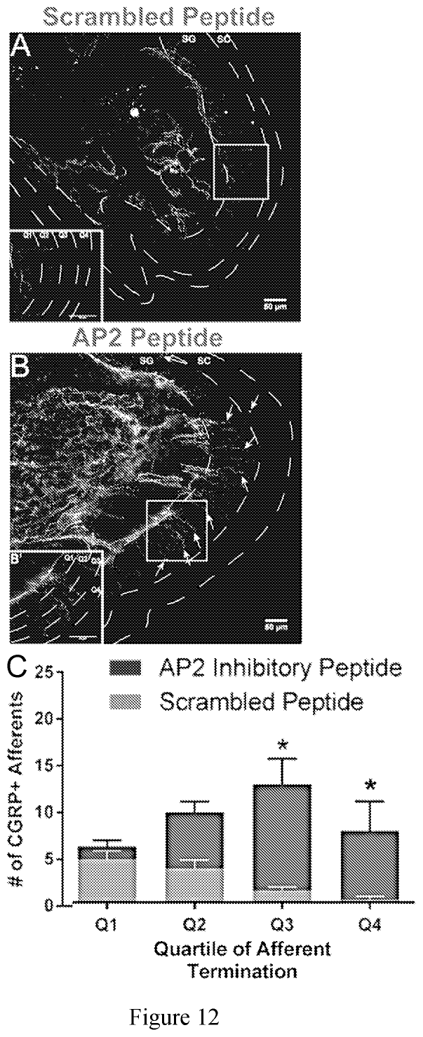

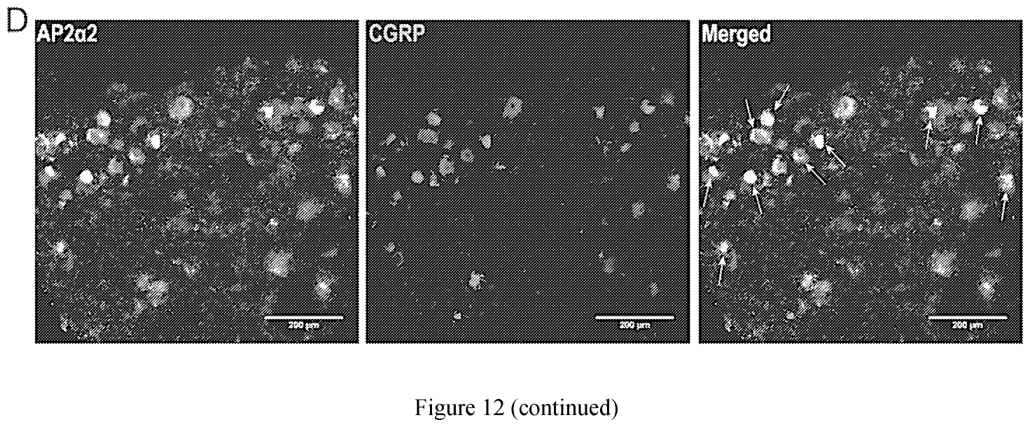

Intraplantar injection of the AP2 inhibitor peptide caused nociceptor CGRP retention within the superficial layers of the epidermis: Peripheral nociceptor afferents were previously shown to terminate in structurally distinct tissue layers in the dermis and epidermis. Specifically, CGRP⁺ nociceptor afferents were shown to terminate in the stratum spinosum layer. Localized inhibition of endocytosis for 24 hours, under non-inflammatory conditions, resulted in the visualization of CGRP immunoreactivity in the very distal layers of the stratum granulosum (SG) (n=3 mice), indicating decreased CGRP tonal release (FIG. 12). These data suggest that CGRP nociceptor afferents actually extend far more superficially in the dermis than previously thought. However, CGRP retention in peripheral fibers was not observed in animals that were administered the AP2 inhibitor peptide 24 hours after the establishment of CFA-induced inflammation (FIG. 20). Prior studies have shown that release of CGRP from primary afferent neurons is increased during the period of maximal hyperalgesia that accompanies peripheral inflammation and therefore the AP2 inhibitor peptide given 24 hours after CFA might not be expected to alter CGRP immunoreactivity in peripheral terminals. Also observed was granuloma-like clustering of immune cells after incision injury (previously observed following CFA injection) indicating possible alterations in immune cell coordination; however, the Ap2 inhibitory peptide did not appear to interrupt the attraction of immune cells to the site of injury (FIG. 19). The pathophysiological consequences of these granuloma-like artifacts in the pain models used are not currently known but also might be due to decreased CGRP release.

Differential expression of AP2α2 in CGRP⁺ neurons was also observed in human DRG: Human and mouse AP2α2 share ˜98% amino acid identity (data not shown) suggesting a strong evolutionary pressure to preserve protein function. Here we conducted hDRG immunohistochemistry studies probing for AP2α2 and CGRP and we observed that hDRG also exhibited AP2α2 differential expression within CGRP⁺ neurons (FIG. 12D). Therefore, human inflammatory pain likely also depends on AP2α2-mediated nociceptor endocytosis and should lend itself to pharmacological manipulation by the lipidated AP2 inhibitory peptide. All AP2 targeted peptides in this study utilized sequences derived from human proteins (Table 1).

### DISCUSSION

Using a genetic and a pharmacological approach in non-transgenic animals, we have demonstrated that inhibition of extra-synaptic nociceptor endocytosis significantly alters inflammatory pain-like behaviors. Nociceptors were locally targeted and provided long-lasting analgesia opening a new path for the development of future analgesics.

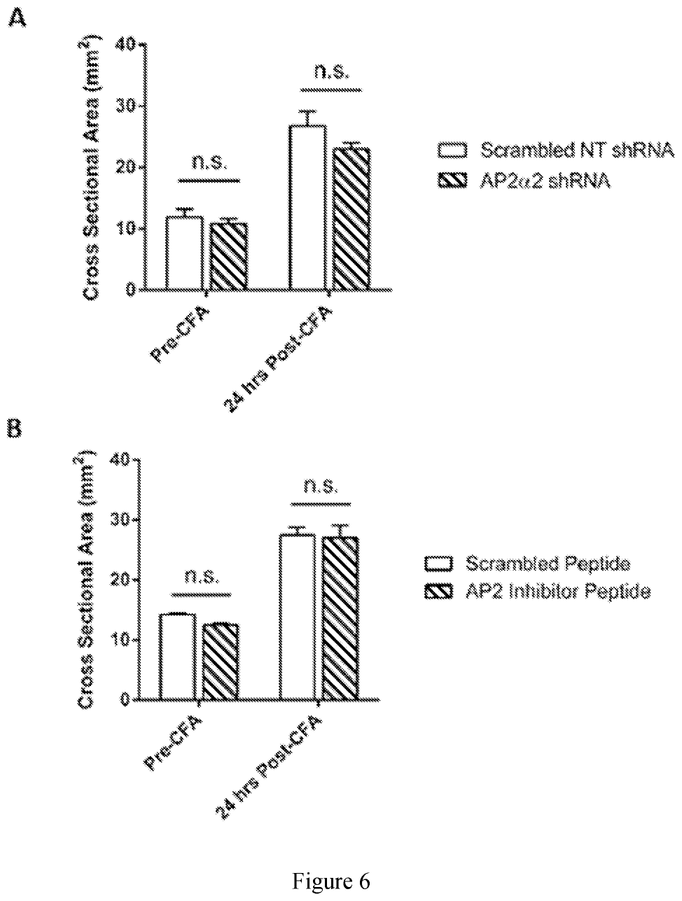

Our characterization of AP2α2 expression in mouse DRG neurons revealed that peptidergic IB4⁻ neurons preferentially express AP2α2 (FIG. 7A). A high level of co-expression with CGRP in human DRG neurons (FIG. 12D) were also observed, suggesting AP2α2 participates in CGRP⁺ nociceptor signaling. CGRP⁺ nociceptors package neuropeptides in large-dense core vesicles (LDCV). They are released in a Ca²⁺-dependent manner, potentiating inflammation, nociception, and immune cell activation. Also LDCVs can fully collapse upon fusion to the membrane. A robust membrane retrieval mechanism, namely endocytosis, would be required after neuropeptide release to allow further LDCV release. The mechanism for AP2-CME in synaptic vesicular membrane retrieval is well-established recycling membrane after synaptic vesicle release. The preferential expression of the extra-synaptic AP2α2 in IB4⁻ neurons is likely due to a specific dependence of membrane retrieval after LDCV release that occurs outside of the synapse. Prominent CGRP immunoreactivity in the SG layer of the dermis following AP2 inhibitory peptide injection (FIG. 6) suggested that changes in pain-like behaviors can be partially attributed to disruptions in CGRP release mechanisms. Decoupling endocytosis from exocytosis, either through the genetic or pharmacological means employed, should have disrupted membrane homeostasis and negatively impacted membrane-localized receptor signaling (i.e. TrkA), ion channel trafficking, and peptidergic signaling. As a result, animals displayed strong attenuation of pain-like behaviors in models of acute and chronic inflammatory pain (FIGS. 7D, 8, 10, and 11). The observed effects on mechanical sensitivity corroborates previously published research implicating peptidergic neurons in the coordination of mechanical and thermal sensitivity during inflammation. The magnitude of our effects on mechanical sensitivity, however, suggests that CGRP release from peripheral neurons indirectly contributes to the development of mechanical sensitivity.

Furthermore, accumulation of membrane-localized K_{Na} channels may also contribute to the observed changes in pain-like behaviors. Previously obtained evidence that inhibition of neuronal endocytosis resulted in the membrane retention of large-conductance Kcnt1 (Slack) K_{Na} channels, which caused the lack of PKA-induced hyperexcitability in cultured DRG neurons. In acutely dissociated neurons from AP2α2 in vivo knockdown, we also found a lack of PKA-induced hyperexcitability (FIG. 6C). Persistent exocytosis of LDCVs without accompanying endocytosis could also cause an increase in membrane Kcnt2 (Slick) channels, another large-conductance K_{Na} channel that was shown localized to CGRP containing LDCVs. We observed a significant reduction in CFA-induced thermal sensitivity after AP2 inhibitory peptide injection (FIG. 10B, C), indicating that blocking ongoing endocytosis, even after full-blown neurogenic inflammation (FIG. 20), altered neuronal excitability. Indeed, overexpression of Kcnt2 channels in DRG neurons was shown to blunt action potential formation.

Using an antigenic lipidated peptidomimetic (HA-peptide) we showed molecular partitioning in both non-inflammatory (FIG. 9A) and inflammatory (FIG. 9B) conditions. The HA-peptide was used as a proxy to understand how small lipidated peptides penetrate into neuronal afferent endings. Both conditions showed HA-immunoreactivity in the dermis, whereas the epidermis and muscle tissue appeared devoid of signal. These findings suggest that either the lipidated peptide was rapidly cleared from these compartments in the hind paw, or the hydrophilic extracellular matrix prevented peptide penetration. Behavioral testing showed that after a single injection, lipidated peptides have a longevity in vivo that was similar to the one observed for the HA-peptide in vitro (FIG. 14). The longevity of small lipidated peptides might depend on membrane turnover kinetics. Injection of our lipidated AP2 inhibitory peptide is aligned with current clinical administration of other FDA-approved lipidated peptides. For example, dulaglutide (Trulicity®) and semaglutide (Ozempic®) are different lipidated Glucagon-like peptide 1 (GLP-1) peptides subcutaneously injected (sometimes daily) to treat diabetes. The GLP-1 peptide (˜30 amino acids) is considerably larger than the peptides described here. Also, in order to achieve systemic absorption and prolonged stability the GLP-1 peptide is administered at 100-1000 fold higher than the concentrations of the lipidated peptides administered to rodents. Locally targeting peripheral nerve afferents at doses that would have minimal systemic absorption is envisioned. However, dosing of both AP2 inhibitor peptides and peptides that target Nav1.8 channels (FIG. 18) require further exploration.

Also uncovered was sex-dependent pain-like behaviors in two different models of inflammatory pain and in two species of animals. These results could only have been evidenced because of the long-lasting pharmacological inhibition of CGRP'⁰ nociceptors and the implementation of an exponential decay best-fit model to interpret thermal responsiveness. This allowed us to characterize the efficacy of our Ap2 inhibitor peptide and quantify recovery kinetics. However, the sex-dependent difference depended upon whether the AP2 inhibitor peptide was given before inflammation develops or after inflammation was already established. After CFA-induced inflammation, control peptide-injected male and female animals exhibited prolonged thermal hyperalgesia that recovered over many days. Injection of the AP2 inhibitor peptide resulted in a rapid recovery of thermal sensitivity in male animals, whereas female animals showed a delayed response. Moreover, τ—values in females for control and AP2 inhibitor peptides were similar. The comparison of recovery kinetics might have indicated that the AP2 inhibitor was ineffective for female animals, however, A.U.C. analysis revealed that analgesia was provided for both sexes (FIGS. 10F and 10G). In contrast, in the incisional pain model, where the AP2 inhibitor peptide was given prior to the development of injury and inflammation, recovery τ—values considerably differed between the control and AP2 inhibitor peptides, irrespective of gender (FIGS. 111 and 11J).

In post-incisional models of pain, prior research found a lack of sex-differences in mechanical sensitivity and hot plate assessment. By using the Hargreaves's method to assess discreet unilateral thermal responsiveness, we observed a sexual dimorphic pain response. In this model, however, a 6-hour hind paw pre-injection procedure was included (FIGS. 11C, 11D, 11I, 11J; FIG. 17). Control male animals exhibited typical pain behaviors following hind paw incision: a significant increase in thermal sensitivity followed by a near-linear recovery phase. Female thermal sensitivity was less than males' (FIG. 17), almost significant (p=0.08). It is considered that if more animals were tested these data would have achieved statistical significance. Moreover, female control animals displayed a unique behavioral profile following hind paw incision: a plateau of thermal hyperalgesia that did not resolve until 7 days post-surgery (FIG. 11D). The recovery was best fitted, not by a one phase, but by a two-phase decay equation (data not shown), suggesting that thermal hyperalgesia by incisional pain in females utilizes multiple processes compared to males. Male rats had a faster recovery time constant after incision (FIGS. 11I,J) which is likely the result of peripherally-based mechanisms. In contrast, female rats seem to have a physiological system optimized for maintaining persistent thermal sensitivity once it begins. This would suggest recruitment of both peripheral and central mechanisms. There are sex-differences in the CGRP receptor components within trigeminal ganglion, medulla and the spinal cord despite equivalent levels of CGRP mRNA. Through the long-term inhibition of CGRP⁺ nociceptors, gender differences in how thermal hyperalgesia is manifested were uncovered. These data are consistent with the idea that there are sex-differences in both the prevalence and the intensity of chronic inflammatory and post-operative pain in humans.

Local administration of therapeutics targeting peripheral nociceptor afferents is becoming a more preferable approach to treat pain because it decreases side effects, including addiction. For example, local injection of reformulated anesthetics is a current alternative to opioids use for pain relief. However, two major challenges remain for locally applied drugs: specificity and duration of action. For patients with ongoing injury-associated pain and associated inflammation, targeting specifically the TRPV1/CGRP⁺ class of afferent fibers may be key in providing effective pain relief. Here, we have demonstrated using lipidated peptidomimetics, specific and long-lasting reduction in pain behaviors, positioning these types of molecules as a new class of analgesics.

Although the present disclosure has been described with respect to one or more particular embodiments and/or examples, it will be understood that other embodiments and/or examples of the present disclosure may be made without departing from the scope of the present disclosure.

1. A peptide comprising the following sequence:
(SEQ ID NO: 7)
X¹X²X³X⁴LX⁵
wherein
X¹ is chosen from D, E, S, and T;
X², X³, and X⁴ are independently chosen from any amino acid; and
X⁵ is chosen from L and I; and
wherein L, X¹, and/or X⁵ is optionally phosphorylated and the peptide is 6-20 amino acid residues long.

2. The peptide of claim 1, wherein the C-terminal amino acid residue or the amino acid residue immediately preceding the C-terminal amino acid is phosphorylated.

3. The peptide of claim 1, wherein the peptide is lipidated.

4. The peptide of claim 3, wherein the lipidation is at the N-terminal amino acid residue.

5. The peptide of claim 3, wherein the lipidation is myristoylation, octanoylation, lauroylation, palmitoylation, or stearoylation.

6. The peptide of claim 1, wherein the peptide has the following sequence: X⁶X¹X²X³X⁴LX⁵ (SEQ ID NO:8), wherein X⁶ is chosen from S and T, and X⁶ is optionally phosphorylated.

7. The peptide of claim 1, comprising a sequence chosen from SEQ ID NOs:1, 2, 3, 4, 5, 8, 9, 10, 11, and 12.

8. A composition comprising one or more peptide of claim 1 and a pharmaceutically acceptable carrier.

9. The composition of claim 8, further comprising one or more analgesic agent and/or one or more anesthetic agent.

10. The composition of claim 8, wherein the one or more analgesic and/or the one or more anesthetic agent is acetaminophen, aspirin, ibuprofen, naproxen, meloxicam, ketorolac, diclofenac, ketoprofen, piroxicam, metamizole, bupivacaine, etidocaine, levobupivacaine, lidocaine, mepivacaine, prilocaine, ropivacaine, procaine, chloroprocaine, hydrocortisone, triamcinolone, methylprednisolone, or a combination thereof.

11. The composition of claim 8, further comprising AP2-CME targeting shRNA and/or AP2-CME targeting siRNA.

12. A method of treating pain or increasing pain sensitivity in a subject in need of treatment comprising:
administering to the subject in need of treatment a therapeutically effective amount of one or more composition of claim 8,
wherein pain of the subject in need of treatment is ameliorated or the pain sensitivity of the subject in need of treatment is increased.

13. The method of claim 12, further comprising administering one or more analgesic agent and/or one or more anesthetic agent.

14. The method of claim 12, wherein the administration step is performed in anticipation of pain.

15. The method of claim 12, wherein the subject in need of treatment has an injury, a chronic disease, a chronic inflammation, Morton's neuroma, operative/post-operative pain or a combination thereof.

16. The method of claim 15, wherein the injury is a spinal cord injury, a nerve injury, a burn, or a combination thereof.

17. The method of claim 16, wherein the chronic disease is diabetes, Herpes zoster, major depressive disorder, fibromyalgia, migraine, arthritis, amyotrophic lateral sclerosis, multiple sclerosis, inflammatory bowel disease, schizophrenia, autism spectrum disorders, cancer, radiculopathy, or a combination thereof.

18. The method of claim 12, wherein the peptide administered to the subject has a sequence chosen from SEQ ID NOs:1, 2, 3, 4, 5, 8, 9, 10, 11, 12, and combinations thereof.

19. The method of claim 12, wherein the subject's pain is ameliorated for 1-120 hours following a single administration step.

20. The method of claim 12, wherein the subject's pain is ameliorated for 24-120 hours following a single administration step.
